(19) United States
(12) Patent Application Publication
(10) Pub. No.: US 2024/0299335 A1
(43) Pub. Date: Sep. 12, 2024

(54) SHELF-STABLE FORMULATIONS OF BENZOYL PEROXIDE AND METHODS OF PRODUCING SAME

(71) Applicant: BLUE HILL TECHNOLOGIES LLC, New Haven, CT (US)

(72) Inventors: David LIGHT, Branford, CT (US); Kaury KUCERA, Orange, CT (US); Jill NAILOR, Mundelein, IL (US)

(21) Appl. No.: 18/666,047

(22) Filed: May 16, 2024

Related U.S. Application Data
(63) Continuation of application No. PCT/US2023/015111, filed on Mar. 13, 2023.
(60) Provisional application No. 63/324,950, filed on Mar. 29, 2022.
(60) Provisional application No. 63/319,674, filed on Mar. 14, 2022.

(51) Int. Cl. A61K 31/327; A61D 7/00; A61J 3/00; A61K 45/06; A61K 47/10; A61K 47/20; A61K 47/22

(52) U.S. Cl. CPC A61K 31/327; A61D 7/00; A61J 3/00; A61K 45/06; A61K 47/10; A61K 47/20; A61K 47/22

(57) ABSTRACT

Disclosed herein are shelf-stable formulations comprising benzoyl peroxide in a formulation effective to reduce or prevent degradation of the benzoyl peroxide to benzene. Further provided herein are methods of preventing or reducing degradation of benzoyl peroxide to benzene in a drug product, as well as methods of identifying drug products containing benzoyl peroxide that form high levels of benzene.

### CROSS-REFERENCE

This application is a continuation of International Patent Application No. PCT/US2023/015111, filed Mar. 13, 2023, which claims the benefit of U.S. Provisional Application No. 63/319,674, filed Mar. 14, 2022, and U.S. Provisional Application No. 63/324,950, filed Mar. 29, 2022, each of which is incorporated herein by reference in its entirety.

### BACKGROUND

Benzoyl peroxide is a drug that is commonly used to treat skin conditions, such as acne vulgaris. Benzoyl peroxide may decompose to benzene under certain circumstances. Benzene is both carcinogenic and a germ cell mutagen. The United States Food & Drug Administration (FDA) and other regulatory agencies worldwide currently restrict the levels of benzene in drug products to 2 parts per million (ppm) or less. Drug products currently approved by the FDA containing benzoyl peroxide ostensibly do not contain or do not form benzene levels in amounts that violate regulations set by the FDA and other regulatory agencies throughout the world.

### SUMMARY

The disclosure provided herein arises from the unexpected and surprising discovery that drug products containing benzoyl peroxide currently approved by regulatory agencies, including the FDA, and sold commercially to treat skin conditions form unacceptably high levels of benzene when stored under certain conditions. The disclosure provided herein addresses the considerable unmet need for providing shelf-stable formulations containing benzoyl peroxide that do not contain and do not form levels of benzene that exceed acceptable limits set by the FDA and other regulatory agencies throughout the world, as well as methods for stabilizing benzoyl peroxide formulations and for identifying drug products that have unacceptably high levels of benzene.

In one aspect, a shelf-stable formulation is provided comprising: an effective amount of benzoyl peroxide in a formulation effective to reduce or prevent degradation of the benzoyl peroxide to benzene, wherein, when the shelf-stable formulation is subjected to stability testing following regulatory agency approval guidelines (e.g., according to “Guidance for Industry Q1A(R2) Stability Testing of New Drug Substances and Products”, Revision 2, November 2003), the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 37° C. for 7 days or more, a concentration of benzene in the shelf-stable formulation is less than about 2 parts per million (2 ppm). In some cases, when the shelf-stable formulation is heated to 37° C. for 7 days or more, the concentration of benzene in the shelf-stable formulation is less than about 1 ppm. In some cases, when the shelf-stable formulation is heated to 37° C. for 7 days or more, the concentration of benzene in the shelf-stable formulation is less than about 0.5 ppm. In some cases, when the shelf-stable formulation is heated to 37° C. for 7 days or more, the concentration of benzene in the shelf-stable formulation is less than about 0.1 ppm. In some cases, when the shelf-stable formulation is heated to 37° C. for 7 days or more, the concentration of benzene in the shelf-stable formulation is undetectable. In some cases, when the shelf-stable formulation is heated to 37° C. for 8 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 37° C. for 10 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 37° C. for 12 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 37° C. for 14 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 37° C. for 7 days or more, at a humidity of from 25% to 80%, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 50° C. for 7 days or more, the concentration of benzene in the shelf-stable formulation is less than about 2 ppm. In some cases, when the shelf-stable formulation is heated to 50° C. for 7 days or more, the concentration of benzene in the shelf-stable formulation is less than about 1 ppm. In some cases, when the shelf-stable formulation is heated to 50° C. for 7 days or more, the concentration of benzene in the shelf-stable formulation is less than about 0.5 ppm. In some cases, when the shelf-stable formulation is heated to 50° C. for 7 days or more, the concentration of benzene in the shelf-stable formulation is less than about 0.1 ppm. In some cases, when the shelf-stable formulation is heated to 50° C. for 7 days or more, the concentration of benzene in the shelf-stable formulation is undetectable. In some cases, when the shelf-stable formulation is heated to 50° C. for 8 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 50° C. for 10 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 50° C. for 12 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 50° C. for 14 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 50° C. for 7 days or more, at a humidity of from 25% to 80%, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 70° C. for 7 days or more, the concentration of benzene in the shelf-stable formulation is less than about 2 ppm. In some cases, when the shelf-stable formulation is heated to 70° C. for 7 days or more, the concentration of benzene in the shelf-stable formulation is less than about 1 ppm. In some cases, when the shelf-stable formulation is heated to 70° C. for 7 days or more, the concentration of benzene in the shelf-stable formulation is less than about 0.5 ppm. In some cases, when the shelf-stable formulation is heated to 70° C. for 7 days or more, the concentration of benzene in the shelf-stable formulation is less than about 0.1 ppm. In some cases, when the shelf-stable formulation is heated to 70° C. for 7 days or more, the concentration of benzene in the shelf-stable formulation is undetectable. In some cases, when the shelf-stable formulation is heated to 70° C. for 8 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 70° C. for 10 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 70° C. for 12 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 70° C. for 14 days, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is heated to 70° C. for 7 days or more, at a humidity of from 25% to 80%, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following Current Good Manufacturing Practice (CGMP) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following International Council for Harmonisation of Technical Requirements for Pharmaceuticals for Human Use (ICH) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following European Medicines Agency (EMA) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following United States Food & Drug Administration (FDA) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following guidelines from a regulatory agency in which the shelf-stable formulation is to be registered or sold, the concentration of benzene in the shelf-stable formulation is less than about 2 ppm, less than about 1 ppm, less than about 0.5 ppm, less than about 0.1 ppm, undetectable, or any combination thereof. In some cases, the shelf-stable formulation comprises at least one stabilizing agent. In some cases, the shelf-stable formulation comprises a plurality of stabilizing agents. In some cases, the at least one stabilizing agent or the plurality of stabilizing agents is present in the shelf-stable formulation in an amount effective to prevent or reduce degradation of the benzoyl peroxide to benzene. In some cases, the at least one stabilizing agent or the plurality of stabilizing agents is present in the shelf-stable formulation in an amount effective to prevent a concentration of benzene in the shelf-stable formulation from exceeding about 2 ppm, exceeding about 1 ppm, exceeding about 0.5 ppm, exceeding about 0.1 ppm, or any combination thereof. In some cases, the at least one stabilizing agent or the plurality of stabilizing agents is or comprises a gas or a plurality of gases. In some cases, the gas or the plurality of gases is dissolved in the shelf-stable formulation. In some cases, the gas or the plurality of gases is or comprises a noble gas, a medical gas, or both. In some cases, the gas or the plurality of gases is or comprises hydrogen, hydrogen sulfide, carbon monoxide, ozone, nitric oxide, helium, neon, argon, krypton, xenon, or any combination thereof. In some cases, the at least one stabilizing agent or the plurality of stabilizing agents is or comprises the shelf-stable formulation or substantially all of the shelf-stable formulation excluding the benzoyl peroxide and any additional active ingredient. In some cases, the at least one stabilizing agent or the plurality of stabilizing agents is or comprises an antioxidant. In some cases, the antioxidant is or comprises a small molecule. In some cases, the antioxidant is or comprises a biomolecule. In some cases, the antioxidant is or comprises an antioxidant selected from the group consisting of: a metal oxide, a metal nanoparticle, a transition metal oxide, a nano-antioxidant, and any combination thereof. In some cases, the antioxidant is selected from the group consisting of: silicon dioxide, copper oxide, zinc oxide, magnesium oxide, titanium dioxide, cerium oxide, iron oxide, molybdenum trioxide, lutetium oxide, and any combination thereof. In some cases, the antioxidant is or comprises tert-Butylhydroxyquinone (TBHQ). In some cases, the antioxidant is or comprises butylated hydroxytoluene (BHT). In some cases, the shelf-stable formulation further comprises at least one solvent. In some cases, the at least one solvent is present in the shelf-stable formulation in an amount effective to prevent or reduce degradation of the benzoyl peroxide to benzene. In some cases, the at least one solvent is present in the shelf-stable formulation in an amount effective to prevent a concentration of benzene in the shelf-stable formulation from exceeding about 2 ppm, exceeding about 1 ppm, exceeding about 0.5 ppm, exceeding about 0.1 ppm, or any combination thereof. In some cases, the solvent is water. In some cases, the shelf-stable formulation further comprises a surfactant. In some cases, the surfactant is or comprises a detergent, a wetting agent, an emulsifier, a foaming agent, a dispersant, or any combination thereof. In some cases, the shelf-stable formulation further comprises a substance that increases the viscosity of the shelf-stable formulation. In some cases, the substance that increases the viscosity of the shelf-stable formulation is a thickener, a gelling agent, or both. In some cases, the benzoyl peroxide, the at least one stabilizing agent or the plurality of stabilizing agents, the at least one solvent, or any combination thereof is encapsulated. In some cases, the benzoyl peroxide, the at least one stabilizing agent or the plurality of stabilizing agents, the at least one solvent, or any combination thereof is encapsulated in a plurality of particles. In some cases, the plurality of particles comprises silica, metal oxides, hydrogels, liposomes, or any combination thereof. In some cases, the plurality of particles are heat stable. In some cases, the no less than about 95%, no less than about 98%, no less than about 99%, no less than about 99.9%, or no less than about 99.99% of the plurality of particles remain structurally unaffected and/or do not release encapsulated material when heated to 37° C. for 7 days or more, when heated to 50° C. for 7 days or more, when heated to 70° C. for 7 days or more, when subjected to stability testing in accordance with a regulatory agency guidelines, or any combination thereof. In some cases, the plurality of particles have an average diameter selected from the group consisting of: less than about 10 mm, less than about 1 mm, less than about 100 μm, less than about 10 μm, less than about 1 μm, less than about 100 nm, less than about 10 nm, and less than about 1 nm. In some cases, the particles are modified to open and release benzoyl peroxide when contacting a target. In some cases, the target is acne vulgaris. In some cases, the target is a biological marker on the surface of acne vulgaris. In some cases, the target is a lipid, a protein, a sugar, or a polysaccharide on the surface of acne vulgaris. In some cases, the benzoyl peroxide, the at least one stabilizing agent, the at least one solvent, or any combination thereof, is encapsulated with a purity selected from the group consisting of: greater than about 90%, greater than about 95%, greater than about 97%, greater than about 98%, greater than about 99%, and greater than about 99.9%. In some cases, no more than about 0.1%, no more than about 1%, no more than about 2%, no more than about 3%, no more than about 4%, no more than about 5%, or no more than about 10% of the benzoyl peroxide, the at least one stabilizing agent, the at least one solvent, or any combination thereof is not encapsulated. In some cases, no more than about 0.1%, no more than about 1%, no more than about 2%, no more than about 3%, no more than about 4%, no more than about 5%, or no more than about 10% of the benzoyl peroxide, the at least one stabilizing agent, the at least one solvent, or any combination thereof is not encapsulated after heating the shelf-stable formulation to 37° C. for 7 days or more, after heating the shelf-stable formulation to 50° C. for 7 days or more, after heating the shelf-stable formulation to 70° C. for 7 days or more, or after subjecting the shelf-stable formulation to stability testing in accordance with a regulatory agency guidelines. In some cases, the benzoyl peroxide has an average particle diameter smaller than the average diameter of the plurality of particles. In some cases, a pH of the shelf-stable formulation is effective to prevent or reduce degradation of the benzoyl peroxide to benzene. In some cases, a pH of the shelf-stable formulation is effective to prevent a concentration of benzene in the shelf-stable formulation from exceeding about 2 ppm, exceeding about 1 ppm, exceeding about 0.5 ppm, exceeding about 0.1 ppm, or any combination thereof. In some cases, the pH of the shelf-stable formulation is about 7 or less. In some cases, the pH of the shelf-stable formulation is about 5 or less. In some cases, the pH of the shelf-stable formulation is about 3 or less. In some cases, the effective amount of the benzoyl peroxide is from about 2.5% w/v to about 10% w/v. In some cases, the effective amount of the benzoyl peroxide is about 2.5% w/v. In some cases, the effective amount of the benzoyl peroxide is about 2.7% w/v. In some cases, the effective amount of the benzoyl peroxide is about 3% w/v. In some cases, the effective amount of the benzoyl peroxide is about 4% w/v. In some cases, the effective amount of the benzoyl peroxide is about 4.4% w/v. In some cases, the effective amount of the benzoyl peroxide is about 4.5% w/v. In some cases, the effective amount of the benzoyl peroxide is about 5% w/v. In some cases, the effective amount of the benzoyl peroxide is about 5.3% w/v. In some cases, the effective amount of the benzoyl peroxide is about 6% w/v. In some cases, the effective amount of the benzoyl peroxide is about 7% w/v. In some cases, the effective amount of the benzoyl peroxide is about 7.5% w/v. In some cases, the effective amount of the benzoyl peroxide is about 8% w/v. In some cases, the effective amount of the benzoyl peroxide is about 9.8% w/v. In some cases, the effective amount of the benzoyl peroxide is about 10% w/v. In some cases, the effective amount of the benzoyl peroxide is an amount effective to treat a disease or disorder in a subject. In some cases, the effective amount of the benzoyl peroxide is an amount effective to treat a skin condition in a subject. In some cases, the subject is a human. In some cases, the skin condition is acne vulgaris. In some cases, the skin condition is selected from the group consisting of: rosacea, folliculitis, pseudo-folliculitis barbae, progressive macular hypomelanosis, decubitis ulcers, perforating disease, and pitted keratolysis. In some cases, the effective amount of the benzoyl peroxide is an amount effective for veterinary use on a non-human animal. In some cases, the non-human animal is a dog, a cat, or a horse. In some cases, the effective amount of the benzoyl peroxide is an amount effective to treat a skin condition in the non-human animal. In some cases, the skin condition is selected from the group consisting of: pyotraumatic dermatitis, seborrhea, canine acne, and Demodectic mange. In some cases, the effective amount of the benzoyl peroxide is an amount effective to treat the fur or hair of the non-human animal. In some cases, the shelf-stable formulation further comprises one or more active ingredients. In some cases, the one or more active ingredients comprises an antibiotic. In some cases, the antibiotic is or comprises clindamycin, erythromycin, sulfur, or any combination thereof. In some cases, the one or more active ingredients comprises a retinoid or a retinoid-like compound. In some cases, the retinoid or retinoid-like compound is or comprises adapalene, tretinoin, or both. In some cases, the one or more active ingredients comprises an anti-inflammatory agent. In some cases, the anti-inflammatory agent is or comprises hydrocortisone, salicylic acid, niacinamide, or any combination thereof. In some cases, the one or more active ingredients comprises a diuretic. In some cases, the diuretic is spironolactone. In some cases, the shelf-stable formulation is a topical formulation for application to the skin of a subject. In some cases, the shelf-stable formulation is a formulation for application to the hair of a subject. In some cases, the shelf-stable formulation is a lotion, a cream, a gel, a jelly, a foam, a pad, a wipe, a solution, a bar, or a liquid. In some cases, the shelf-stable formulation is a face lotion, a face cream, a face gel, a face mask, a face wash, a soap, a foam cleanser, a face pad, a cleansing bar, a shaving cream, an emollient foam, a cleansing liquid, an exfoliant, a shampoo, or a hydrating wash.

In another aspect, a method of preparing a formulation or a drug product comprising benzoyl peroxide that does not have or does not produce high levels of benzene is provided, the method comprising: a) providing or obtaining a formulation or a drug product comprising benzoyl peroxide identified as having or producing a concentration of benzene greater than about 2 parts per million (ppm); and b) formulating or re-formulating the formulation or the drug product in a shelf-stable formulation that prevents or reduces the formation of benzene; c) packaging or re-packaging the formulation or the drug product in a packaging that prevents or reduces the formation of benzene; d) providing instructions to store the formulation or the drug product under conditions that prevent or reduce the formation of benzene; or e) any combination thereof. In some cases, the formulation or the drug product is a formulation or a drug product approved for use on a human by a regulatory agency. In some cases, the formulation or the drug product is a formulation or a drug product approved to treat a skin condition in a human by a regulatory agency. In some cases, the skin condition is acne vulgaris. In some cases, the formulation or the drug product is for veterinary use on a non-human animal. In some cases, the formulation or the drug product is a topical formulation of the benzoyl peroxide. In some cases, the formulation or the drug product further comprises one or more active ingredients. In some cases, the one or more active ingredients comprises an antibiotic. In some cases, the antibiotic is or comprises clindamycin, erythromycin, sulfur, or any combination thereof. In some cases, the one or more active ingredients comprises a retinoid or a retinoid-like compound. In some cases, the retinoid or retinoid-like compound is or comprises adapalene, tretinoin, or both. In some cases, the one or more active ingredients comprises an anti-inflammatory agent. In some cases, the anti-inflammatory agent is or comprises hydrocortisone, salicylic acid, niacinamide, or any combination thereof. In some cases, the benzoyl peroxide is present in the formulation or the drug product at an amount from about 2.5% w/v to about 10% w/v. In some cases, the benzoyl peroxide is present in the formulation or the drug product at an amount of about 2.5% w/v. In some cases, the benzoyl peroxide is present in the formulation or the drug product at an amount of about 2.7% w/v. In some cases, the benzoyl peroxide is present in the formulation or the drug product at an amount of about 3% w/v. In some cases, the benzoyl peroxide is present in the formulation or the drug product at an amount of about 4% w/v. In some cases, the benzoyl peroxide is present in the formulation or the drug product at an amount of about 4.4% w/v. In some cases, the benzoyl peroxide is present in the formulation or the drug product at an amount of about 4.5% w/v. In some cases, the benzoyl peroxide is present in the formulation or the drug product at an amount of about 5% w/v. In some cases, the benzoyl peroxide is present in the formulation or the drug product at an amount of about 5.3% w/v. In some cases, the benzoyl peroxide is present in the formulation or the drug product at an amount of about 6% w/v. In some cases, the benzoyl peroxide is present in the formulation or the drug product at an amount of about 7% w/v. In some cases, the benzoyl peroxide is present in the formulation or the drug product at an amount of about 7.5% w/v. In some cases, the benzoyl peroxide is present in the formulation or the drug product at an amount of about 8% w/v. In some cases, the benzoyl peroxide is present in the formulation or the drug product at an amount of about 9.8% w/v. In some cases, the benzoyl peroxide is present in the formulation or the drug product at an amount of about 10% w/v. In some cases, the drug product is selected from the group consisting of: 111MedCo®, Acne-Clear®, AcneFree®, Acne Wipeout®, Acnigel®, Anti Bac®, Asepxia®, Ben-Aqua®, Benzac®, Benzagel®, Benzashave®, BenzEFoam®, Benziq®, Benzoil®, Bioelements®, Binora®, Botanic Tree®, Brevoxyl®, CeraVe®, Claridad®, Clean & Clear®, Clear By Design®, Clearasil®, Clearplex®, Clearskin®, Clinac BPO®, CVS®, CVS Health®, Daylogic®, Del-Aqua®, Dermaquest®, Desquam®, Differin®, Dr. Song®, Dr. Zenovia®, Epionce®, Equate®, Equate Beauty®, Ethexderm BPW®, Fostex®, Glo Skin Beauty®, Glossier®, Glytone®, Harris Pharmaceutical®, Humane®, Inova®, Jan Marini Skin Research®, Johnson&Johnson®, Kate Sommerville®, Kroger®, La Roche-Posay®, Lavoclen®, Loroxide®, MDacne®, NeoBenz®, Neutrogena®, Oscion®, Oxy 10®, Pacnex®, PanOxyl®, Paula's Choice®, PCA Skin®, Peroderm®, Persa-Gel®, Peroxin A®, Perrigo®, Persa-Gel®, Proactiv®, ProactivMD®, Proactiv+®, Proactiv Solution®, RB Health®, Replenix Acne®, Rugby Laboratories®, SalSphere®, Salvona®, Seba-Gel®, Signature Caret, Skin Bar MD®, SLMD Sandra Lee MD®, SMACNE®, Solimo®, Soluclenz®, Sonya Dakar®, Taro Pharmaceuticals®, Theroxide®, Touch®, Triaz®, Tula Skincare®, Up&Up®, Vanoxide®, Vivant Skincare®, Walgreens®, Zaclir®, Zapzyt®, Zeroxin®, Zit Stick®, ZoDerm®, Acanya®, Bencort®, Benzaclin®, Benzamycin®, Duac®, Epiduo®, Face Up®, NuOx(®, Sulfoxyl®, and Vanoxide-HC®. In some cases, the formulating or the re-formulating of b) comprises adding at least one stabilizing agent or a plurality of stabilizers to the formulation or the drug product in an amount effective to reduce or prevent degradation of the benzoyl peroxide to benzene. In some cases, the formulating or the re-formulating of b) comprises adding at least one stabilizing agent or a plurality of stabilizing agents to the formulation or the drug product in an amount effective to prevent a concentration of benzene in the shelf-stable formulation from exceeding about 2 ppm. In some cases, the at least one stabilizing agent or the plurality of stabilizing agents is or comprises a gas or a plurality of gases. In some cases, the gas or the plurality of gases is dissolved in the shelf-stable formulation. In some cases, the gas or the plurality of gases is or comprises a noble gas, a medical gas, or both. In some cases, the gas or the plurality of gases is or comprises hydrogen, hydrogen sulfide, carbon monoxide, ozone, nitric oxide, helium, neon, argon, krypton, xenon, or any combination thereof. In some cases, the at least one stabilizing agent or the plurality of stabilizing agents is or comprises the shelf-stable formulation or substantially all of the shelf-stable formulation excluding the benzoyl peroxide and any additional active ingredient. In some cases, the at least one stabilizing agent or the plurality of stabilizing agents is or comprises an antioxidant. In some cases, the antioxidant is or comprises a small molecule. In some cases, the antioxidant is or comprises a biomolecule. In some cases, the antioxidant is or comprises an antioxidant selected from the group consisting of: a metal oxide, a metal nanoparticle, a transition metal oxide, a nano-antioxidant, and any combination thereof. In some cases, the antioxidant is or comprises an antioxidant selected from the group consisting of: silicon dioxide, copper oxide, zinc oxide, magnesium oxide, titanium dioxide, cerium oxide, iron oxide, molybdenum trioxide, lutetium oxide, and any combination thereof. In some cases, the antioxidant is or comprises tert-Butylhydroxyquinone (TBHQ). In some cases, the antioxidant is or comprises butylated hydroxytoluene (BHT). In some cases, the formulating or the re-formulating of b) comprises adding at least one solvent to the formulation or the drug product in an amount effective to reduce or prevent degradation of the benzoyl peroxide to benzene. In some cases, the formulating or the re-formulating of b) comprises adding at least one solvent to the formulation or the drug product in an amount effective to prevent a concentration of benzene in the shelf-stable formulation from exceeding about 2 ppm. In some cases, the solvent is water. In some cases, the formulating or the re-formulating of b) comprises adding stabilized benzoyl peroxide to the formulation or the drug product, wherein the stabilized benzoyl peroxide exhibits reduced or no degradation to benzene as compared to non-stabilized benzoyl peroxide. In some cases, the formulating or the re-formulating of b) comprises adding stabilized benzoyl peroxide to the formulation or the drug product in an amount such that a concentration of benzene in the shelf-stable formulation does not exceed about 2 ppm. In some cases, the stabilized benzoyl peroxide is stabilized by encapsulation. In some cases, the stabilized benzoyl peroxide, the at least one stabilizing agent, the at least one solvent, or any combination thereof is encapsulated. In some cases, the stabilized benzoyl peroxide, the at least one stabilizing agent, the at least one solvent, or any combination thereof is encapsulated in a plurality of particles. In some cases, the plurality of particles comprises silica, metal oxides, hydrogels, liposomes, or any combination thereof. In some cases, the plurality of particles have an average diameter selected from the group consisting of: less than about 10 mm, less than about 1 mm, less than about 100 μm, less than about 10 μm, less than about 1 μm, less than about 100 nm, less than about 10 nm, and less than about 1 nm. In some cases, the particles are modified to open and release benzoyl peroxide when contacting a target. In some cases, the target is acne vulgaris. In some cases, the target is a biological marker on the surface of acne vulgaris. In some cases, the target is a lipid, a protein, a sugar, or a polysaccharide on the surface of acne vulgaris. In some cases, the stabilized benzoyl peroxide, the at least one stabilizing agent, the at least one solvent, or any combination thereof, is encapsulated with a purity selected from the group consisting of: greater than about 90%, greater than about 95%, greater than about 97%, greater than about 98%, greater than about 99%, and greater than about 99.9%. In some cases, no more than about 0.1%, no more than about 1%, no more than about 2%, no more than about 3%, no more than about 4%, no more than about 5%, or no more than about 10% of the stabilized benzoyl peroxide, the at least one stabilizing agent, the at least one solvent, or any combination thereof is not encapsulated. In some cases, the stabilized benzoyl peroxide has an average particle diameter smaller than the average diameter of the plurality of particles. In some cases, the formulating or the re-formulating of b) comprises formulating or re-formulating the benzoyl peroxide at a pH effective to reduce or prevent degradation of the benzoyl peroxide to benzene. In some cases, the formulating or the re-formulating of b) comprises formulating or re-formulating the benzoyl peroxide at a pH effective to prevent a concentration of benzene in the shelf-stable formulation from exceeding about 2 ppm. In some cases, the pH is about 7 or less. In some cases, the pH is about 5 or less. In some cases, the pH is about 3 or less. In some cases, the packaging or the re-packaging of c) comprises packaging or re-packaging the formulation or the drug product in a packaging that prevents or reduces exposure of the benzoyl peroxide to radiation. In some cases, the packaging or the re-packaging of c) comprises packaging or re-packaging the formulation or the drug product in a packaging that prevents or reduces exposure of the benzoyl peroxide to light. In some cases, the packaging or the re-packaging of c) comprises packaging or re-packaging the formulation or the drug product in a packaging that prevents or reduces exposure of the benzoyl peroxide to heat. In some cases, the packaging or the re-packaging of c) comprises packaging or re-packaging the formulation or the drug product in a packaging that prevents or reduces fluctuations in temperature. In some cases, the packaging or the re-packaging of c) comprises packaging or re-packaging the formulation or the drug product in a packaging that prevents a concentration of benzene in the shelf-stable formulation from exceeding about 2 ppm. In some cases, the providing instructions of d) comprises providing instructions to store the formulation or the drug product away from exposure to light. In some cases, the providing instructions of d) comprises providing instructions to store the formulation or the drug product at a temperature at or below about 25° C. In some cases, the providing instructions of d) comprises providing instructions to store the formulation or the drug product at a temperature at or below about 15° C. In some cases, the providing instructions of d) comprises providing instructions to store the formulation or the drug product at a temperature at or below about 10° C. In some cases, the providing instructions of d) comprises providing instructions to store the formulation or the drug product at a temperature at or below about 4° C. In some cases, the providing instructions of d) comprises providing instructions to store the formulation or the drug product at a temperature at or below about 0° C. In some cases, the providing instructions of d) comprises providing instructions to store the formulation or the drug product at a temperature at or below about −20° C. In some cases, the providing instructions of d) comprises providing instructions to store the formulation or the drug product at a temperature at or below about −70° C. In some cases, the providing instructions of d) comprises providing instructions on a label on the formulation, the drug product, or an active pharmaceutical ingredient comprising benzoyl peroxide. In some cases, the providing instructions of d) comprises providing instructions on an instruction sheet separate from the label of the drug product. In some cases, the providing instructions of d) comprises providing instructions on a website. In some cases, the providing instructions of d) comprises providing instructions by a physician or healthcare provider. In some cases, the method further comprises, prior to a), testing the formulation or the drug product for the presence of benzene using one or more analytical techniques. In some cases, the one or more analytical techniques is gas chromatography/mass spectrometry (GC-MS), liquid chromatography/mass spectrometry (LC-MS), high-performance liquid chromatography (HPLC), nuclear magnetic resonance (NMR), gas chromatography with flame ionization detection (GC-FID), selected ion flow tube mass spectrometry (SIFT-MS), or any combination thereof. In some cases, the method further comprises, prior to a), identifying the formulation or the drug product as having high levels of benzene when the concentration of benzene present in the formulation or the drug product is higher than about 2 parts per million (ppm). In some cases, the method further comprises, prior to a), heating the formulation or the drug product to a temperature of at least about 37° C. for at least 7 days. In some cases, prior to the heating, the concentration of benzene present in the formulation or the drug product is greater than or equal to about 2 ppm. In some cases, prior to the heating, the concentration of benzene present in the formulation or the drug product is less than about 2 ppm. In some cases, testing the formulation or the drug product further comprises testing the formulation or the drug product for the presence of benzene at a first time point and at a second time point. In some cases, a concentration of benzene in the formulation or the drug product at the second time point is greater than at the first time point. In some cases, at the first time point, the formulation or the drug product has a concentration of benzene that is less than about 2 ppm, and at the second time point, the formulation or the drug product has a concentration of benzene that is greater than about 2 ppm.

In yet another aspect, a formulation or a drug product is provided comprising: a) an effective amount of benzoyl peroxide; and b) instructions or guidance to store the formulation or the drug product under conditions that prevent or reduce degradation of the benzoyl peroxide to benzene. In some cases, the formulation or the drug product further comprises instructions or guidance to store the formulation or the drug product under conditions in which a concentration of benzene in the formulation or the drug product does not exceed about 2 ppm. In some cases, the instructions or guidance are on the label of the formulation or the drug product. In some cases, the instructions or guidance are provided on an instruction sheet separate from the label of the formulation or the drug product. In some cases, the instructions or guidance are provided on a website. In some cases, the instructions or guidance are provided by a physician or healthcare provider. In some cases, the conditions comprise storing the formulation or the drug product away from exposure to light, radiation, heat, or any combination thereof. In some cases, the conditions comprise storing the formulation or the drug product at a temperature at or below about 25° C. In some cases, the conditions comprise storing the formulation or the drug product at a temperature at or below about 15° C. In some cases, the conditions comprise storing the formulation or the drug product at a temperature at or below about 10° C. In some cases, the conditions comprise storing the formulation or the drug product at a temperature at or below about 4° C. In some cases, the conditions comprise storing the formulation or the drug product at a temperature at or below about 0° C. In some cases, the conditions comprise storing the formulation or the drug product at a temperature at or below about −20° C. In some cases, the conditions comprise storing the formulation or the drug product at a temperature at or below about −70° C. In some cases, the effective amount of the benzoyl peroxide is in a shelf-stable formulation of any one of the preceding. In some cases, the formulation or the drug product is in a packaging effective to reduce or prevent degradation of the benzoyl peroxide to benzene. In some cases, the formulation or the drug product is in a packaging effective to prevent a concentration of benzene from exceeding about 2 ppm.

In yet another aspect, a formulation or a drug product is provided comprising: a) an effective amount of benzoyl peroxide; and b) packaging containing the effective amount of benzoyl peroxide, wherein the packaging reduces or prevents degradation of the benzoyl peroxide to benzene. In some cases, the packaging prevents a concentration of benzene in the formulation or drug product from exceeding about 2 ppm. In some cases, the packaging prevents or reduces exposure of the benzoyl peroxide to radiation. In some cases, the packaging prevents or reduces exposure of the benzoyl peroxide to light. In some cases, the packaging prevents or reduces exposure of the benzoyl peroxide to heat. In some cases, the packaging prevents or reduces fluctuations in temperature.

In some cases, the effective amount of the benzoyl peroxide is in a shelf-stable formulation of any one of the preceding. In some cases, the shelf-stable formulation of any one of the preceding further comprises packaging containing the effective amount of benzoyl peroxide, wherein the packaging reduces or prevents degradation of the benzoyl peroxide to benzene. In some cases, the shelf-stable formulation of any one of the preceding further comprises packaging effective to prevent a concentration of benzene in the shelf-stable formulation from exceeding about 2 ppm. In some cases, the shelf-stable formulation of any one of the preceding further comprises instructions or guidance to store the formulation or the drug product under conditions that prevent or reduce degradation of the benzoyl peroxide to benzene. In some cases, the shelf-stable formulation of any one of the preceding further comprises instructions or guidance to store the formulation or the drug product under conditions such that a concentration of benzene in the shelf-stable formulation does not exceed about 2 ppm.

In another aspect, a drug lot is provided comprising: a plurality of drug products, each drug product comprising an effective amount of benzoyl peroxide, wherein no more than about 5% of the plurality of drug products produce greater than about 2 parts per million (ppm) benzene when the drug products are heated to about 37° C. for 7 days or more. In some cases, no more than about 1% of the plurality of drug products produce greater than about 2 parts per million (ppm) benzene when the drug products are heated to about 37° C. for 7 days or more. In some cases, no more than about 0.5% of the plurality of drug products produce greater than about 2 parts per million (ppm) benzene when the drug products are heated to about 37° C. for 7 days or more. In some cases, no more than about 0.1% of the plurality of drug products produce greater than about 2 parts per million (ppm) benzene when the drug products are heated to about 37° C. for 7 days or more. In some cases, no more than about 5%, no more than about 1%, no more than about 0.5%, or no more than about 0.1% of the plurality of drug products produce greater than about 2 parts per million (ppm) benzene when the drug products are subjected to stability testing in accordance with guidelines from a regulatory agency.

In another aspect, a kit is provided comprising: (a) a first container or a plurality of first containers containing therein a shelf-stable drug product or formulation comprising an effective amount of benzoyl peroxide, wherein the benzoyl peroxide is in substantially pure form or is in a formulation effective to reduce or prevent degradation of the benzoyl peroxide to benzene; (b) a second container or a plurality of second containers containing therein one or more ingredient; and (c) instructions for combining the shelf-stable drug product or formulation with the one or more ingredient to generate a compounded benzoyl peroxide drug product, wherein, when the shelf-stable drug product or formulation is heated to 37° C. for 7 days or more, a concentration of benzene in the shelf-stable drug product or formulation is less than about 2 parts per million (2 ppm). In some cases, when the compounded benzoyl peroxide drug product is heated to 37° C. for 7 days or more, a concentration of benzene in the compounded benzoyl peroxide drug product is less than about 2 ppm. In some cases, the one or more ingredient is selected from the group consisting of: a formulation, an inactive ingredient, an active ingredient other than benzoyl peroxide, a solvent, a stabilizing agent, a capsule, a particle, and any combination thereof. In some cases, the compounded benzoyl peroxide drug product is for use by an individual within a period of time after generation of the compounded benzoyl peroxide drug product. In some cases, the period of time is selected from the group consisting of: 36 months or less, 24 months or less, 18 months or less, 12 months or less, 9 months or less, 6 months or less, 3 months or less, 90 days or less, 60 days or less, 45 days or less, 30 days or less, 15 days or less, 10 days or less, 7 days or less, 5 days or less, 72 hours or less, 48 hours or less, 24 hours or less, 12 hours or less, 1 hour or less, 30 minutes or less, 10 minutes or less, and 5 minutes or less. In some cases, the compounded benzoyl peroxide drug product is for use by an animal. In some cases, the benzoyl peroxide has a purity selected from the group consisting of: greater than about 90%, greater than about 95%, greater than about 97%, greater than about 98%, greater than about 99%, and greater than about 99.9%. In some cases, the benzoyl peroxide is in the form of a crystalline powder. In some cases, when the compounded benzoyl peroxide drug product is subjected to stability testing following guidelines from a regulatory agency in a jurisdiction in which the compounded benzoyl peroxide drug product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof. In some cases, the kit further comprises packaging which prevents or reduces the degradation of benzoyl peroxide to benzene. In some cases, the kit further comprises instructions to store the first container or the plurality of first containers, the second container or the plurality of second containers, and/or the compounded benzoyl peroxide drug product at a temperature selected from the group consisting of: at or below about 25° C., at or below about 20° C., at or below about 15° C., at or below about 10° C., at or below about 4° C., at or below about 0° C., at or below about −20° C., at or below about −70° C., and any combination thereof. In some cases, when the shelf-stable drug product or formulation is subjected to stability testing following guidelines from a regulatory agency in a jurisdiction in which the kit is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof. In some cases, when contents of the first container, contents of the second container, the first container or plurality of first containers, the second container or plurality of second containers, or any combination thereof, is subjected to stability testing following guidelines from a regulatory agency in a jurisdiction in which the kit is to be registered or sold, the concentration of benzene is less than about 2 ppm, less than about 1 ppm, less than about 0.5 ppm, less than about 0.1 ppm, undetectable, or any combination thereof.

In yet another aspect, a compounded benzoyl peroxide product is provided comprising: (a) an effective amount of benzoyl peroxide, wherein the benzoyl peroxide is in substantially pure form or is in a formulation effective to reduce or prevent degradation of the benzoyl peroxide to benzene; and (b) one or more ingredient. In some cases, the compounded benzoyl peroxide drug product is for use by an individual within a period of time after generation of the compounded benzoyl peroxide drug product. In some cases, the period of time is selected from the group consisting of: 36 months or less, 24 months or less, 18 months or less, 12 months or less, 9 months or less, 6 months or less, 3 months or less, 90 days or less, 60 days or less, 45 days or less, 30 days or less, 15 days or less, 10 days or less, 7 days or less, 5 days or less, 72 hours or less, 48 hours or less, 24 hours or less, 12 hours or less, 1 hour or less, 30 minutes or less, 10 minutes or less, and 5 minutes or less. In some cases, when the compounded benzoyl peroxide drug product is heated to 37° C., 50° C., or 70° C. for 7 days or more, a concentration of benzene in the compounded benzoyl peroxide drug product is less than about 2 ppm. In some cases, the one or more ingredient is selected from the group consisting of: a formulation, an inactive ingredient, an active ingredient other than benzoyl peroxide, a solvent, a stabilizing agent, a capsule, a particle, and any combination thereof. In some cases, the compounded benzoyl peroxide drug product is for use by an animal. In some cases, the benzoyl peroxide has a purity selected from the group consisting of: greater than about 90%, greater than about 95%, greater than about 97%, greater than about 98%, greater than about 99%, and greater than about 99.9%. In some cases, the benzoyl peroxide is in the form of a crystalline powder. In some cases, when the compounded benzoyl peroxide drug product is subjected to stability testing following guidelines from a regulatory agency in a jurisdiction in which the compounded benzoyl peroxide drug product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof. In some cases, the compounded benzoyl peroxide product further comprises packaging which prevents or reduces the degradation of benzoyl peroxide to benzene. In some cases, the compounded benzoyl peroxide product further comprises instructions to store the compounded benzoyl peroxide drug product at a temperature selected from the group consisting of: at or below about 25° C., at or below about 20° C., at or below about 15° C., at or below about 10° C., at or below about 4° C., at or below about 0° C., at or below about −20° C., at or below about −70° C., and any combination thereof. In some cases, when the compounded benzoyl peroxide product is subjected to stability testing following guidelines from a regulatory agency in a jurisdiction in which the compounded benzoyl peroxide product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof.

In another aspect, a method of producing a compounded benzoyl peroxide product of any one of the preceding is provided, the method comprising: mixing or combining the benzoyl peroxide with the one or more ingredient, thereby producing the compounded benzoyl peroxide product.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a proposed degradation pathway of benzoyl peroxide to benzene.

FIG. 2 depicts benzene levels (ppm) formed in various FDA-approved drug products containing benzoyl peroxide after incubation at 50° C. for three weeks.

FIG. 3 depicts the effects of various antioxidants on benzene levels (ppm) in a 10% w/w benzoyl peroxide formulation after incubation at 37° C. for two weeks.

FIG. 4 depicts the effects of various antioxidants on benzene levels (ppm) in a 10% w/w benzoyl peroxide formulation after incubation at 50° C. for two weeks.

FIG. 5 depicts the effects of different antioxidants on benzene levels (ppm) in two different FDA-approved drug products containing benzoyl peroxide, after incubation at 50° C. for 2 days.

FIG. 6 depicts the effects of various solvents on benzene levels (ppm) in a 10% w/w benzoyl peroxide formulation after incubation at 37° C. for two weeks.

FIG. 7 depicts the effects of various solvents on benzene levels (ppm) in a 10% w/w benzoyl peroxide formulation after incubation at 50° C. for two weeks.

FIG. 8 depicts the effects of various solvents on benzene levels (ppm) in a 10% benzoyl peroxide formulation after incubation at 37° C. for 10 days.

FIG. 9 depicts the effects of pH on benzene levels (ppm) in a 10% benzoyl peroxide formulation after incubation at 37° C. for 10 days.

FIG. 10 depicts the effects of pH on benzene levels (ppm) in a 10% benzoyl peroxide formulation after incubation at 50° C. for 10 days.

FIG. 11 depicts benzene levels (ppm) formed when crystalline benzoyl peroxide is incubated at 50° C. for 12 days.

FIG. 12 depicts benzene levels (ppm) formed when a commercially-available encapsulated benzoyl peroxide is incubated at 50° C. for 8 days.

FIG. 13 depicts benzene levels (ppm) formed when four commercially-available benzoyl peroxide products were incubated at 50° C. for 14 days either in their native formulation, or with modifications to increase stability by increasing water content and reducing pH.

### DETAILED DESCRIPTION OF THE DISCLOSURE

This disclosure is based on the unexpected and surprising discovery that drug products approved by regulatory agencies, including the FDA, containing benzoyl peroxide form unacceptably high levels of benzene under various conditions, and often contain unacceptably high levels of benzene when sold commercially. The disclosure provided herein addresses the considerable unmet need for shelf-stable formulations of benzoyl peroxide that do not form levels of benzene that exceed acceptable levels set by the FDA and other regulatory agencies throughout the world, as well as methods for stabilizing formulations containing benzoyl peroxide and for identifying drug products comprising benzoyl peroxide as having unacceptably high levels of benzene.

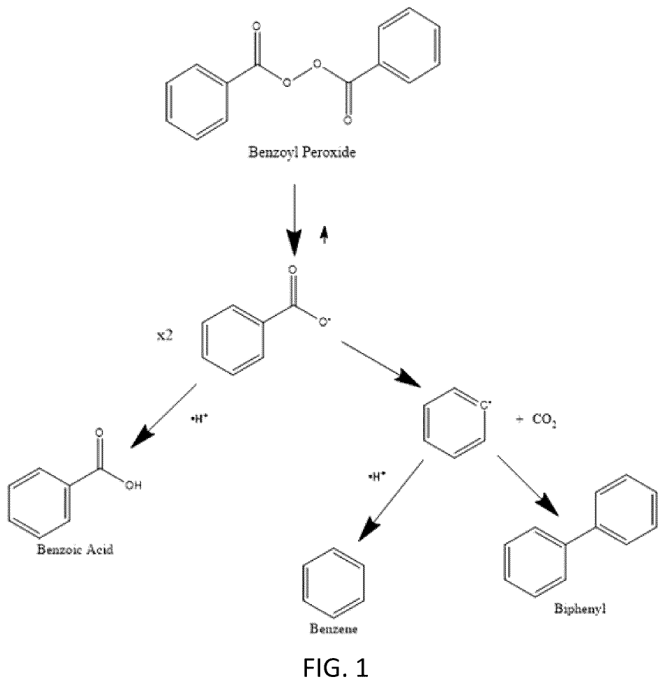

Benzoyl peroxide may, under certain conditions, degrade or decompose to various degradation products. FIG. 1 depicts a schematic of a degradation pathway of benzoyl peroxide to the degradation products: benzoic acid, benzene, and biphenyl. Benzene is a known carcinogen and the levels of benzene allowed in drug products is tightly regulated. For example, the FDA limits the amount of benzene in drug products to 2 ppm or potentially lower, similar to other regulatory agencies throughout the world. Unexpectedly and surprisingly, current FDA-approved drug products containing benzoyl peroxide (e.g., topical formulations of benzoyl peroxide to treat skin conditions, e.g., acne vulgaris) currently sold on the market contain or produce high levels of benzene, including levels of benzene that, in some cases, are unacceptable for a regulated drug product.

Provided herein are shelf-stable formulations of benzoyl peroxide that do not form unacceptably high levels of benzene (e.g., levels of benzene that exceed 2 ppm). Further provided herein are shelf-stable formulations of benzoyl peroxide that do not form levels of benzene that exceed levels approved for use in a drug product by a regulatory agency (e.g., the FDA). Further provided herein are methods of identifying drug products containing benzoyl peroxide that form unacceptably high levels of benzene and methods of stabilizing formulations and drug products containing benzoyl peroxide.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise.

As used herein, the term “about” a number refers to that number plus or minus 10% of that number. The term “about” a range refers to that range minus 10% of its lowest value and plus 10% of its greatest value.

As used herein, the terms “subject”, “individual”, and “patient” are used interchangeably herein. None of the terms are to be interpreted as requiring the supervision of a medical professional (e.g., a doctor, nurse, physician's assistant, orderly, hospice worker). As used herein, a subject may be any animal, including mammals (e.g., a human or non-human animal) and non-mammals. In one embodiment, the subject is a human.

The terms “consumer” and “end user” are used interchangeably herein and refer to an individual or entity that purchases, uses, and/or stores a product as described herein (e.g., a treatment, a drug product, a formulation, or an ingredient containing benzoyl peroxide) in accordance with common practice or with a provided instruction. In some cases, a consumer or end user is an individual that has been prescribed or has been instructed to take or use (e.g., by a physician) a drug product (e.g., containing benzoyl peroxide) as provided herein (e.g., for the treatment of a skin condition, e.g., acne vulgaris). In some cases, a consumer or end user is an individual that purchases a product provided herein (e.g., a drug product containing benzoyl peroxide) from e.g., a store, a pharmacy, a hospital, and the like. In some cases, a consumer or end user is an individual that uses a product provided herein (e.g., a drug product containing benzoyl peroxide) for the on-label use or the off-label use of the drug product. In some cases, a consumer is a purchaser, such as a retailer, a wholesaler, and the like. In some cases, a consumer is a shipper, a packager, a labeler, and the like. In some cases, a consumer is any individual or entity involved in any part of the pharmaceutical supply chain.

As used herein, the terms “treat”, “treating”, or “treatment,” and other grammatical equivalents, include ameliorating or preventing the underlying causes of one or more symptoms of a disease or condition; alleviating, abating, or ameliorating one or more symptoms of a disease or condition; ameliorating, preventing, or reducing the appearance, severity, or frequency of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically.

The term “drug product” as used herein, refers to an agent, compound, composition, or formulation capable of being administered to a subject and containing an active ingredient (e.g., benzoyl peroxide) which relieves, to some extent, one or more of the symptoms of the disease or condition being treated (e.g., reduce Propionibacterium acnes, reduce inflammatory acne lesions, reduce noninflammatory acne lesions.), or reduces the underlying cause of the disease or condition being treated (e.g., acne vulgaris). In some cases, the drug product is approved by a regulatory agency (e.g., the United States Food and Drug Administration (FDA), the European Medicines Agency (EMA), Japan's Pharmaceutical and Medical Devices Agency (PMDA), Health Canada, the Swiss Agency for Therapeutic Products (Swissmedic), and Australia's Therapeutic Goods Administration (TGA)) (e.g., for the treatment of acne vulgaris). In some cases, the drug product is commercially available. In some cases, the drug product contains one or more additional active ingredients (e.g., benzoyl peroxide combined with an antibiotic, an anti-inflammatory agent, or a retinoid).

The terms “effective amount” or “therapeutically effective amount” are used interchangeably herein and refer to a sufficient amount of an agent or a compound being administered which relieves, to some extent, one or more of the symptoms of the disease or condition being treated, or reduces the underlying cause of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms or underlying cause of the disease (e.g., without undue adverse side effects). In some embodiments, an appropriate “effective amount” in any individual case is determined using techniques, such as a dose escalation study. The term “therapeutically effective amount” includes, for example, a prophylactically effective amount. An “effective amount” of a compound disclosed herein may be an amount effective to achieve a desired effect or therapeutic improvement (e.g., without undue adverse side effects). An “effective amount” of a compound disclosed herein may be an amount effective to achieve one or more desired outcomes. It should be understood that, in some cases, “an effective amount” or “a therapeutically effective amount” varies from subject to subject, due to variation in metabolism of the composition, age, weight, general condition of the subject, concomitant medications the subject may be taking, the condition being treated, the severity of the condition being treated, and the judgment of the prescribing physician.

In some cases, the disease or condition being treated is a skin condition (e.g., acne vulgaris). The underlying cause of the skin condition may be, e.g., bacteria underneath the skin. In some cases, the compositions may be used to treat and/or prevent an underlying cause of a skin condition, e.g., by killing bacteria underneath the skin, as well as by helping pores shed dead skin cells and excess sebum. In some cases, the drug product is being used on-label (e.g., acne vulgaris) or in accordance with manufacturer-provided instructions. In some cases, the drug product is being used off-label to treat a skin condition (e.g., rosacea, folliculitis, pseudo-folliculitis barbae, progressive macular hypomelanosis, decubitis ulcers, perforating disease, and pitted keratolysis).

In various aspects, the formulations and drug products provided herein contain an amount of benzoyl peroxide that is effective to treat a condition (e.g., a skin condition, such as acne vulgaris). In various aspects, the benzoyl peroxide is present in the formulations and drug products provided herein at a concentration that, when applied or administered to a subject (e.g., topically), is effective to treat a condition (e.g., a skin condition, such as acne vulgaris). In some cases, the benzoyl peroxide is present in the formulations and drug products provided herein in an amount of from about 2.5% w/v to about 10% w/v.

The terms “package” and “packaging” are used interchangeably herein and refer to any manner of storing, holding, containing, distributing, dispensing, and/or preserving a drug product (e.g., ajar, a vial, a container, a box, an ampoule, a phial, a bottle, a tube, a flask, a repository, a case, a holder, a canister, a vessel, and the like).

The term “unacceptably high”, when used in the context of benzene, refers to a level (e.g., concentration) (e.g., about 2 ppm or greater) of benzene that exceeds acceptable limits set by a regulatory agency (e.g., the FDA) for a drug product.

### Shelf-Stable Formulations and Drug Products of the Disclosure

Provided herein are shelf-stable formulations and drug products comprising an effective amount of benzoyl peroxide (e.g., present in an amount (e.g., concentration) effective to treat one or more conditions (e.g., skin condition)). Generally, the shelf-stable formulations and drug products provided herein are in a formulation that prevents or reduces the degradation and/or decomposition of benzoyl peroxide to benzene. In some cases, the shelf-stable formulations and drug products provided herein contain one or more stabilizing agents as described herein. In some cases, the shelf-stable formulations and drug products provided herein are packaged in packaging that prevents or reduces the degradation and/or decomposition of benzoyl peroxide to benzene. In some cases, the shelf-stable formulations and drug products provided herein are provided with instructions or guidance to store the shelf-stable formulations and drug products under conditions that prevent or reduce the degradation and/or decomposition of benzoyl peroxide to benzene.

Generally, the formulations provided herein are shelf-stable formulations of benzoyl peroxide. The term “shelf-stable” as used herein refers to a formulation containing benzoyl peroxide that, when placed under certain conditions (e.g., standard storage conditions, increased temperature, stability testing, etc.), does not produce or form unacceptably high levels of benzene. A “shelf-stable formulation” can refer to a formulation containing benzoyl peroxide which does not have or does not produce unacceptably high levels of benzene, particularly when stored in a storage area (e.g., a shelf, a storage room, a delivery truck, a cargo hold, a room, a closet, a cabinet, a cupboard, and the like) (e.g., under standard storage conditions). In some cases, benzene levels are considered to be unacceptably high when they exceed about 2 ppm. In some cases, benzene levels are considered to be unacceptably high when they exceed a level deemed by a regulatory agency (e.g., the FDA) to be acceptable for use in a drug product.

In one aspect, provided herein are shelf-stable formulations comprising an effective amount of benzoyl peroxide in a formulation effective to reduce or prevent degradation of the benzoyl peroxide to benzene.

In some cases, when the shelf-stable formulation is heated to 37° C. for 7 days or more, a concentration of benzene in the shelf-stable formulation is less than about 2 ppm. In some cases, when the shelf-stable formulation is heated to 37° C. for 7 days or more, a concentration of benzene in the shelf-stable formulation is less than about 1 ppm. In some cases, when the shelf-stable formulation is heated to 37° C. for 7 days or more, a concentration of benzene in the shelf-stable formulation is less than about 0.5 ppm. In some cases, when the shelf-stable formulation is heated to 37° C. for 7 days or more, a concentration of benzene in the shelf-stable formulation is less than about 0.1 ppm. In some cases, when the shelf-stable formulation is heated to 37° C. for 7 days or more, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 37° C. for 8 days, a concentration of benzene in the shelf-stable formulation is less than about 2 ppm. In some cases, when the shelf-stable formulation is heated to 37° C. for 8 days, a concentration of benzene in the shelf-stable formulation is less than about 1 ppm. In some cases, when the shelf-stable formulation is heated to 37° C. for 8 days, a concentration of benzene in the shelf-stable formulation is less than about 0.5 ppm. In some cases, when the shelf-stable formulation is heated to 37° C. for 8 days, a concentration of benzene in the shelf-stable formulation is less than about 0.1 ppm. In some cases, when the shelf-stable formulation is heated to 37° C. for 8 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 37° C. for 10 days, a concentration of benzene in the shelf-stable formulation is less than about 2 ppm. In some cases, when the shelf-stable formulation is heated to 37° C. for 10 days, a concentration of benzene in the shelf-stable formulation is less than about 1 ppm. In some cases, when the shelf-stable formulation is heated to 37° C. for 10 days, a concentration of benzene in the shelf-stable formulation is less than about 0.5 ppm. In some cases, when the shelf-stable formulation is heated to 37° C. for 10 days, a concentration of benzene in the shelf-stable formulation is less than about 0.1 ppm. In some cases, when the shelf-stable formulation is heated to 37° C. for 10 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 37° C. for 12 days, a concentration of benzene in the shelf-stable formulation is less than about 2 ppm. In some cases, when the shelf-stable formulation is heated to 37° C. for 12 days, a concentration of benzene in the shelf-stable formulation is less than about 1 ppm. In some cases, when the shelf-stable formulation is heated to 37° C. for 12 days, a concentration of benzene in the shelf-stable formulation is less than about 0.5 ppm. In some cases, when the shelf-stable formulation is heated to 37° C. for 12 days, a concentration of benzene in the shelf-stable formulation is less than about 0.1 ppm. In some cases, when the shelf-stable formulation is heated to 37° C. for 12 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 37° C. for 14 days, a concentration of benzene in the shelf-stable formulation is less than about 2 ppm. In some cases, when the shelf-stable formulation is heated to 37° C. for 14 days, a concentration of benzene in the shelf-stable formulation is less than about 1 ppm. In some cases, when the shelf-stable formulation is heated to 37° C. for 14 days, a concentration of benzene in the shelf-stable formulation is less than about 0.5 ppm. In some cases, when the shelf-stable formulation is heated to 37° C. for 14 days, a concentration of benzene in the shelf-stable formulation is less than about 0.1 ppm. In some cases, when the shelf-stable formulation is heated to 37° C. for 14 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 37° C. for 7 days or more, at a humidity of from 25% to 80%, a concentration of benzene in the shelf-stable formulation is less than about 2 ppm. In some cases, when the shelf-stable formulation is heated to 37° C. for 7 days or more, at a humidity of from 25% to 80%, a concentration of benzene in the shelf-stable formulation is less than about 1 ppm. In some cases, when the shelf-stable formulation is heated to 37° C. for 7 days or more, at a humidity of from 25% to 80%, a concentration of benzene in the shelf-stable formulation is less than about 0.5 ppm. In some cases, when the shelf-stable formulation is heated to 37° C. for 7 days or more, at a humidity of from 25% to 80%, a concentration of benzene in the shelf-stable formulation is less than about 0.1 ppm. In some cases, when the shelf-stable formulation is heated to 37° C. for 7 days or more, at a humidity of from 25% to 80%, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 7 days or more, a concentration of benzene in the shelf-stable formulation is less than about 2 ppm. In some cases, when the shelf-stable formulation is heated to 50° C. for 7 days or more, a concentration of benzene in the shelf-stable formulation is less than about 1 ppm. In some cases, when the shelf-stable formulation is heated to 50° C. for 7 days or more, a concentration of benzene in the shelf-stable formulation is less than about 0.5 ppm. In some cases, when the shelf-stable formulation is heated to 50° C. for 7 days or more, a concentration of benzene in the shelf-stable formulation is less than about 0.1 ppm. In some cases, when the shelf-stable formulation is heated to 50° C. for 7 days or more, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 8 days, a concentration of benzene in the shelf-stable formulation is less than about 2 ppm. In some cases, when the shelf-stable formulation is heated to 50° C. for 8 days, a concentration of benzene in the shelf-stable formulation is less than about 1 ppm. In some cases, when the shelf-stable formulation is heated to 50° C. for 8 days, a concentration of benzene in the shelf-stable formulation is less than about 0.5 ppm. In some cases, when the shelf-stable formulation is heated to 50° C. for 8 days, a concentration of benzene in the shelf-stable formulation is less than about 0.1 ppm. In some cases, when the shelf-stable formulation is heated to 50° C. for 8 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 10 days, a concentration of benzene in the shelf-stable formulation is less than about 2 ppm. In some cases, when the shelf-stable formulation is heated to 50° C. for 10 days, a concentration of benzene in the shelf-stable formulation is less than about 1 ppm. In some cases, when the shelf-stable formulation is heated to 50° C. for 10 days, a concentration of benzene in the shelf-stable formulation is less than about 0.5 ppm. In some cases, when the shelf-stable formulation is heated to 50° C. for 10 days, a concentration of benzene in the shelf-stable formulation is less than about 0.1 ppm. In some cases, when the shelf-stable formulation is heated to 50° C. for 10 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 12 days, a concentration of benzene in the shelf-stable formulation is less than about 2 ppm. In some cases, when the shelf-stable formulation is heated to 50° C. for 12 days, a concentration of benzene in the shelf-stable formulation is less than about 1 ppm. In some cases, when the shelf-stable formulation is heated to 50° C. for 12 days, a concentration of benzene in the shelf-stable formulation is less than about 0.5 ppm. In some cases, when the shelf-stable formulation is heated to 50° C. for 12 days, a concentration of benzene in the shelf-stable formulation is less than about 0.1 ppm. In some cases, when the shelf-stable formulation is heated to 50° C. for 12 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 14 days, a concentration of benzene in the shelf-stable formulation is less than about 2 ppm. In some cases, when the shelf-stable formulation is heated to 50° C. for 14 days, a concentration of benzene in the shelf-stable formulation is less than about 1 ppm. In some cases, when the shelf-stable formulation is heated to 50° C. for 14 days, a concentration of benzene in the shelf-stable formulation is less than about 0.5 ppm. In some cases, when the shelf-stable formulation is heated to 50° C. for 14 days, a concentration of benzene in the shelf-stable formulation is less than about 0.1 ppm. In some cases, when the shelf-stable formulation is heated to 50° C. for 14 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 50° C. for 7 days or more, at a humidity of from about 25% to about 80%, a concentration of benzene in the shelf-stable formulation is less than about 2 ppm. In some cases, when the shelf-stable formulation is heated to 50° C. for 7 days or more, at a humidity of from about 25% to about 80%, a concentration of benzene in the shelf-stable formulation is less than about 1 ppm. In some cases, when the shelf-stable formulation is heated to 50° C. for 7 days or more, at a humidity of from about 25% to about 80%, a concentration of benzene in the shelf-stable formulation is less than about 0.5 ppm. In some cases, when the shelf-stable formulation is heated to 50° C. for 7 days or more, at a humidity of from about 25% to about 80%, a concentration of benzene in the shelf-stable formulation is less than about 0.1 ppm. In some cases, when the shelf-stable formulation is heated to 50° C. for 7 days or more, at a humidity of from about 25% to about 80%, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 7 days or more, a concentration of benzene in the shelf-stable formulation is less than about 2 ppm. In some cases, when the shelf-stable formulation is heated to 70° C. for 7 days or more, a concentration of benzene in the shelf-stable formulation is less than about 1 ppm. In some cases, when the shelf-stable formulation is heated to 70° C. for 7 days or more, a concentration of benzene in the shelf-stable formulation is less than about 0.5 ppm. In some cases, when the shelf-stable formulation is heated to 70° C. for 7 days or more, a concentration of benzene in the shelf-stable formulation is less than about 0.1 ppm. In some cases, when the shelf-stable formulation is heated to 70° C. for 7 days or more, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 8 days, a concentration of benzene in the shelf-stable formulation is less than about 2 ppm. In some cases, when the shelf-stable formulation is heated to 70° C. for 8 days, a concentration of benzene in the shelf-stable formulation is less than about 1 ppm. In some cases, when the shelf-stable formulation is heated to 70° C. for 8 days, a concentration of benzene in the shelf-stable formulation is less than about 0.5 ppm. In some cases, when the shelf-stable formulation is heated to 70° C. for 8 days, a concentration of benzene in the shelf-stable formulation is less than about 0.1 ppm. In some cases, when the shelf-stable formulation is heated to 70° C. for 8 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 10 days, a concentration of benzene in the shelf-stable formulation is less than about 2 ppm. In some cases, when the shelf-stable formulation is heated to 70° C. for 10 days, a concentration of benzene in the shelf-stable formulation is less than about 1 ppm. In some cases, when the shelf-stable formulation is heated to 70° C. for 10 days, a concentration of benzene in the shelf-stable formulation is less than about 0.5 ppm. In some cases, when the shelf-stable formulation is heated to 70° C. for 10 days, a concentration of benzene in the shelf-stable formulation is less than about 0.1 ppm. In some cases, when the shelf-stable formulation is heated to 70° C. for 10 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 12 days, a concentration of benzene in the shelf-stable formulation is less than about 2 ppm. In some cases, when the shelf-stable formulation is heated to 70° C. for 12 days, a concentration of benzene in the shelf-stable formulation is less than about 1 ppm. In some cases, when the shelf-stable formulation is heated to 70° C. for 12 days, a concentration of benzene in the shelf-stable formulation is less than about 0.5 ppm. In some cases, when the shelf-stable formulation is heated to 70° C. for 12 days, a concentration of benzene in the shelf-stable formulation is less than about 0.1 ppm. In some cases, when the shelf-stable formulation is heated to 70° C. for 12 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 14 days, a concentration of benzene in the shelf-stable formulation is less than about 2 ppm. In some cases, when the shelf-stable formulation is heated to 70° C. for 14 days, a concentration of benzene in the shelf-stable formulation is less than about 1 ppm. In some cases, when the shelf-stable formulation is heated to 70° C. for 14 days, a concentration of benzene in the shelf-stable formulation is less than about 0.5 ppm. In some cases, when the shelf-stable formulation is heated to 70° C. for 14 days, a concentration of benzene in the shelf-stable formulation is less than about 0.1 ppm. In some cases, when the shelf-stable formulation is heated to 70° C. for 14 days, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is heated to 70° C. for 7 days or more, at a humidity of from about 25% to about 80%, a concentration of benzene in the shelf-stable formulation is less than about 2 ppm. In some cases, when the shelf-stable formulation is heated to 70° C. for 7 days or more, at a humidity of from about 25% to about 80%, a concentration of benzene in the shelf-stable formulation is less than about 1 ppm. In some cases, when the shelf-stable formulation is heated to 70° C. for 7 days or more, at a humidity of from about 25% to about 80%, a concentration of benzene in the shelf-stable formulation is less than about 0.5 ppm. In some cases, when the shelf-stable formulation is heated to 70° C. for 7 days or more, at a humidity of from about 25% to about 80%, a concentration of benzene in the shelf-stable formulation is less than about 0.1 ppm. In some cases, when the shelf-stable formulation is heated to 70° C. for 7 days or more, at a humidity of from about 25% to about 80%, a concentration of benzene in the shelf-stable formulation is undetectable. In some cases, the concentration of benzene is detected by an analytical system (e.g., gas chromatography/mass spectrometry (GC-MS)).

In some cases, when the shelf-stable formulation is subjected to stability testing following Current Good Manufacturing Practice (CGMP) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following International Council for Harmonisation of Technical Requirements for Pharmaceuticals for Human Use (ICH) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following European Medicines Agency (EMA) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following United States Food & Drug Administration (FDA) guidelines, the concentration of benzene in the shelf-stable formulation is less than about 2 ppm, less than about 1 ppm, less than about 0.5 ppm, less than about 0.1 ppm, undetectable, or any combination thereof. In some cases, when the shelf-stable formulation is subjected to stability testing following guidelines from a regulatory agency in which the shelf-stable formulation is to be registered or sold, the concentration of benzene in the shelf-stable formulation is less than about 2 ppm, less than about 1 ppm, less than about 0.5 ppm, less than about 0.1 ppm, undetectable, or any combination thereof. In some cases, stability testing following guidelines from a regulatory agency are in accordance with “Guidance for Industry Q1A(R2) Stability Testing of New Drug Substances and Products”, Revision 2, November 2003.

In some cases, when the shelf-stable formulation is heated to 37° C. for 7 days or more, a concentration of benzene in the shelf-stable formulation is less than a concentration approved for use in a drug product by a regulatory agency. In some cases, when the shelf-stable formulation is heated to 50° C. for 7 days or more, a concentration of benzene in the shelf-stable formulation is less than a concentration approved for use in a drug product by a regulatory agency. In some cases, when the shelf-stable formulation is heated to 70° C. for 7 days or more, a concentration of benzene in the shelf-stable formulation is less than a concentration approved for use in a drug product by a regulatory agency. In some cases, the concentration approved of benzene for use in a drug product is less than 2 ppm.

In some cases, the regulatory agency is selected from the group consisting of: the United States Food and Drug Administration (FDA), the European Medicines Agency (EMA), Japan's Pharmaceutical and Medical Devices Agency (PMDA), Health Canada, the Swiss Agency for Therapeutic Products (Swissmedic), and Australia's Therapeutic Goods Administration (TGA). In some cases, the regulatory agency is the United States Food and Drug Administration (FDA).

In various aspects, the shelf-stable formulations provided herein contain an effective amount of benzoyl peroxide (e.g., an amount (e.g., concentration) that, when applied or administered to a subject, is effective to treat a disease, a disorder, or a condition (e.g., skin conditions)). In some cases, the shelf-stable formulations and drug products provided herein contain an amount of benzoyl peroxide approved by a regulatory agency (e.g., the FDA) for use in or on a (e.g., human) subject (e.g., to treat a condition (e.g., a skin condition)). In some cases, the subject is a human. In some cases, the amount of benzoyl peroxide present in the shelf-stable formulations and drug products provided herein is an amount effective to treat acne vulgaris in a (e.g., human) subject. In some cases, the amount of benzoyl peroxide present in the shelf-stable formulations and drug products provided herein is an amount effective to treat a skin condition in a (e.g., human) subject, the skin condition selected from the group consisting of: rosacea, folliculitis, pseudo-folliculitis barbae, progressive macular hypomelanosis, decubitis ulcers, perforating disease, and pitted keratolysis.

In some aspects, the effective amount of the benzoyl peroxide is an amount effective for veterinary use on a non-human animal (e.g., dog, cat, livestock (e.g., cow, horse, sheep, pig)). In some cases, the effective amount of the benzoyl peroxide is an amount effective to treat a skin condition in the non-human animal. The skin condition in the non-human animal may be selected from the group consisting of: pyotraumatic dermatitis, seborrhea, canine acne, and Demodectic mange. In some cases, the effective amount of the benzoyl peroxide is an amount effective to treat the fur or hair of the non-human animal.

In some cases, an effective amount of benzoyl peroxide is an amount from about 2.5% w/v to about 10% w/v. In some cases, an effective amount of benzoyl peroxide is about 2.5% w/v. In some cases, an effective amount of benzoyl peroxide is about 2.7% w/v. In some cases, an effective amount of benzoyl peroxide is about 3% w/v. In some cases, an effective amount of benzoyl peroxide is about 4% w/v. In some cases, an effective amount of benzoyl peroxide is about 4.4% w/v. In some cases, an effective amount of benzoyl peroxide is about 4.5% w/v. In some cases, an effective amount of benzoyl peroxide is about 5% w/v. In some cases, an effective amount of benzoyl peroxide is about 5.3% w/v. In some cases, an effective amount of benzoyl peroxide is about 6% w/v. In some cases, an effective amount of benzoyl peroxide is about 7% w/v. In some cases, an effective amount of benzoyl peroxide is about 7.5% w/v. In some cases, an effective amount of benzoyl peroxide is about 8% w/v. In some cases, an effective amount of benzoyl peroxide is about 9.8% w/v. In some cases, an effective amount of benzoyl peroxide is about 10% w/v.

In various aspects, the shelf-stable formulations and drug products provided herein may comprise at least one stabilizing agent or a plurality of stabilizing agents. In some cases, the stabilizing agent is or comprises any agent, molecule, compound, or condition that is effective to prevent or reduce degradation of benzoyl peroxide to benzene. In some cases, the at least one stabilizing agent or the plurality of stabilizing agents is present in the shelf-stable formulations and drug products provided herein in an amount effective to prevent or reduce degradation of benzoyl peroxide to benzene. In some cases, the at least one stabilizing agent or the plurality of stabilizing agents is or comprises any agent, molecule, compound, or condition that is effective to prevent benzene levels from exceeding 2 ppm in the shelf-stable formulations and drug products provided herein. In some cases, the at least one stabilizing agent or the plurality of stabilizing agents is or comprises the shelf-stable formulation or substantially all of the shelf-stable formulation excluding the benzoyl peroxide and any additional active ingredient (e.g., the shelf-stable formulation or drug product may consist of or consist essentially of the stabilizing agent(s) and the benzoyl peroxide (optionally, with one or more additional active ingredient other than the benzoyl peroxide)).

In various aspects, the at least one stabilizing agent or the plurality of stabilizing agents is or comprises a gas or a plurality of gases. The gas or the plurality of gases may be dissolved in the shelf-stable formulations or drug products. The gas or plurality of gases may be or may comprise a noble gas, a medical gas, or both. Non-limiting examples of gases that may be used as stabilizing agents include hydrogen, hydrogen sulfide, carbon monoxide, ozone, nitric oxide, helium, neon, argon, krypton, xenon, and the like.

In various aspects, the at least one stabilizing agent or the plurality of stabilizing agents is or comprises an antioxidant. In some cases, the antioxidant is or comprises a small molecule. In some cases, the antioxidant is or comprises a biomolecule. The antioxidant may be or may comprise an antioxidant selected from the group consisting of: a metal oxide, a metal nanoparticle, a transition metal oxide, a nano-antioxidant, and any combination thereof. Non-limiting examples of metal oxides that may be used to stabilize benzoyl peroxide in the shelf-stable formulations and drug products provided herein include copper oxide, zinc oxide, magnesium oxide, titanium dioxide, cerium oxide, iron oxide, molybdenum trioxide, lutetium oxide, and any combination thereof. In some cases, the antioxidant is selected from the group consisting of: silicon dioxide, vitamin E acetate, eugenol, butylated hydroxyanisole (BHA), butylated hydroxytoluene (BHT), tert-butylhydroxyquinone (TBHQ), vitamin C (ascorbic acid), and any combination thereof. In some cases, the antioxidant is tert-butylhydroxyquinone (TBHQ). In some cases, the antioxidant is butylated hydroxytoluene (BHT). In some cases, the shelf-stable formulations and drug products provided herein comprise a single antioxidant species. In other cases, the shelf-stable formulations and drug products provided herein comprise a plurality of antioxidant species (e.g., a combination of TBHQ and BHT).

In some cases, the antioxidant(s) is/are present in the shelf-stable formulations and drug products provided herein at an amount of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater. In some aspects, the shelf-stable formulations and drug products may consist or consist essentially of the antioxidant and the benzoyl peroxide (and optionally, one or more additional active ingredients other than the benzoyl peroxide).

In some cases, the shelf-stable formulations and drug products provided herein contains or comprises a solvent. In some cases, the solvent is in an amount effective to prevent or reduce degradation of the benzoyl peroxide to benzene. In some cases, the solvent is in an amount effective to prevent or reduce the production of benzene. In some cases, the solvent is present in an amount effective to prevent a concentration of benzene in the shelf-stable formulation from exceeding about 2 ppm. In some cases, the amount of solvent comprises the entire formulation excluding the active ingredient (e.g., benzoyl peroxide) or combination of active ingredients. For example, the formulation may consist of, or consist essentially of, the solvent and one or more active ingredients (e.g., including benzoyl peroxide), wherein the amount of solvent is effective to prevent or reduce the production or formation of benzene. In some cases, the solvent is a stabilizer of the benzoyl peroxide. In some cases, the stabilizer is in an amount effective to prevent or reduce degradation of the benzoyl peroxide to benzene. In some cases, the stabilizer is in an amount effective to prevent or reduce the production of benzene in the formulation. In some cases, the solvent is water. In some cases, the water is in an amount effective to prevent or reduce degradation of the benzoyl peroxide to benzene. In some cases, the water is in an amount effective to prevent or reduce the production of benzene. In some cases, the solvent contains a surfactant (e.g., detergents, wetting agents, emulsifiers, foaming agents, dispersants, etc.), or a plurality of surfactants. In some cases, the surfactant or plurality of surfactants function to better dissolve or disperse the benzoyl peroxide. In some cases, the solvent contains a substance that can increase the viscosity of the liquid (e.g., thickeners, gelling agents, etc.). In some cases, the substance or plurality of substances that increase the viscosity of the liquid function to improve the consumers' experience of using or applying the formulations or drug product.

In some cases, any one of, or any combination of, the benzoyl peroxide, the solvent, and/or the stabilizing agent(s) may be encapsulated (e.g., within a particle). The term “encapsulated” when used herein in context with the components herein, refers to the component (e.g., benzoyl peroxide, solvent, stabilizing agent) surrounded by a shell. Any one of, or any combination of, the benzoyl peroxide, the solvent, and/or the stabilizing agent(s), with or without other ingredients, may be encapsulated to prevent or reduce the degradation of benzoyl peroxide to benzene. In some cases, the encapsulated benzoyl peroxide is isolated from other active ingredients (e.g., prevented from reacting or interacting with other active ingredients) during storage. The encapsulated benzoyl peroxide described herein may be protected from degrading or decomposing to benzene under certain conditions (e.g., such as by heating to 37° C. for a period of time, e.g., 7 days). The encapsulated benzoyl peroxide described herein then is distinguished from currently available encapsulated benzoyl peroxide products that form unacceptably high levels of benzene when exposed to certain conditions (e.g., such as by heating to 37° C. for a period of time; e.g., see Example 7).

In some cases, the shelf-stable formulations and drug products provided herein comprise any one of, or any combination of, the benzoyl peroxide, the solvent, and/or the stabilizing agent(s) encapsulated in a particle. In some cases, other (e.g., inactive) ingredients are added into the shelf-stable formulations and drug products provided herein. In some cases, these other (e.g., inactive) ingredients may also be encapsulated. In some cases, the particle is comprised of silica, metal oxides, hydrogels, liposomes, or any combination thereof.

In some cases, the particles are heat stable. For example, the particles may be substantially unaffected (e.g., structurally) by heating the particles to elevated temperature. In some cases, no less than about 95%, no less than about 98%, no less than about 99%, no less than about 99.9%, or no less than about 99.99% of the particles remain structurally unaffected and/or do not release encapsulated material when heated for a period of time (e.g., to 37° C. for 7 days or more; to 50° C. for 7 days or more; to 70° C. for 7 days or more; when subjected to stability testing according to regulatory agency guidelines, etc.).

In some cases, the particles have an average diameter selected from the group consisting of: less than about 10 mm, less than about 1 mm, less than about 100 μm, less than about 10 μm, less than about 1 μm, less than about 100 nm, less than about 10 nm, and less than about 1 nm. In some cases, any one of, or any combination of, the benzoyl peroxide, the solvent, and/or the stabilizing agent(s) is encapsulated with a purity selected from the group consisting of: greater than about 90%, greater than about 95%, greater than about 97%, greater than about 98%, greater than about 99%, and greater than about 99.9%. In some cases, no more than about 0.1%, no more than about 1%, no more than about 2%, no more than about 3%, no more than about 4%, no more than about 5%, or no more than about 10% of the benzoyl peroxide, the at least one stabilizing agent, the at least one solvent, or any combination thereof is not encapsulated. In some cases, no more than about 0.1%, no more than about 1%, no more than about 2%, no more than about 3%, no more than about 4%, no more than about 5%, or no more than about 10% of the benzoyl peroxide, the at least one stabilizing agent, the at least one solvent, or any combination thereof is not encapsulated after heating the shelf-stable formulation to 37° C. for 7 days or more, after heating the shelf-stable formulation to 50° C. for 7 days or more, after heating the shelf-stable formulation to 70° C. for 7 days or more, or after subjecting the shelf-stable formulation to stability testing in accordance with a regulatory agency guidelines.

In some cases, the particle is a microcapsule. In some cases, the microcapsule has a mean particle size within a range from about 1 μm to about 500 μm. In some cases, the mean particle size is less than about 130 μm, such as less than about 100 μm, and may be within the ranges of about 10 μm to about 130 μm, about 10 μm to about 100 μm, about 20 μm to about 90 μm, about 30 μm to about 70 μm, or about 40 μm to about 60 μm. In some cases, the mean particle size of the microcapsules is from about 30 μm to about 70 μm, for example, about 30 μm, about 40 μm, about 50 μm, about 60 μm, or about 70 μm. In some cases, the particle is a nanocapsule. In some cases, the nanocapsule has a mean particle size within a range from about 1 nm to about 1 μm, for example, from about 1 nm to about 250 nm, from about 1 nm to about 100 nm, from about 1 nm to about 50 nm, from about 10 nm to about 450 nm, from about 10 nm to about 300 nm, from about 10 nm to about 250 nm, from about 10 nm to about 100 nm, from about 10 nm to about 50 nm, from about 100 nm to about 500 nm, from about 100 nm to about 250 nm, from about 250 nm to about 500 nm, from about 250 nm to about 600 nm, from about 250 nm to about 700 nm, from about 250 nm to about 800 nm, from about 250 nm to about 900 nm, from about 500 nm to about 750 nm, or from about 500 nm to about 1 μm. In some cases, the benzoyl peroxide, when encapsulated, has an average particle diameter smaller than the average diameter of the plurality of particles.

Any one of, or any combination of, the benzoyl peroxide, the solvent, and/or the stabilizing agent(s), and optionally, in combination with other ingredients, may be found within the microcapsule core or solubilized in a polymer shell. The wall-forming polymeric material may be a polyacrylate, a polymethacrylate, a cellulose ether or ester, or a combination thereof. In some embodiments, the polymer is a polymethacrylate, such as, but not limited to, poly(methyl methacrylate) co-methacrylic acid (PMMA) or ammonio methacrylate copolymer type B (also known as Eudragit RSPO®). In some cases, the polymer is a cellulose ether or ester such as, but not limited to, methyl cellulose, ethyl cellulose, hydroxypropyl methyl cellulose, hydroxypropyl cellulose, cellulose acetate, cellulose acetate phthalate, hydroxypropyl methyl cellulose acetate phthalate. In some cases, the wall-forming polymeric material is a combination of the aforementioned polymers such as, but not limited to, combinations of ammonio methacrylate copolymer type B (Eudragit RSPO®) with either PMMA or ethyl cellulose (EC).

The microcapsules may comprise one or more additives (e.g., plasticizers, a wax, boron nitride, or a combination thereof). In some cases, the microcapsules contain one or more plasticizers (e.g., tricaprylin (TC), trilaurin (TL), acetyl tributyl citrate (ATBC), acetyl triethyl citrate (ATEC), triethyl citrate (TEC), C12-C15 alkyl benzoate (AB), isopropyl myristate, monoglycerol oleate, tripalmitin, triacetin, or paraffin oil. In another embodiment, the additive is a wax selected from beeswax, abil wax, lanolin, triisostearin, isostearyl isostearate, stearic acid, cetyl alcohol, palmitic acid, glyceryl stearate, or propylene glycol mono palmitostearate (PGMP)). In some cases, the additive is a fatty acid.

In some cases, the particles are modified to open and release benzoyl peroxide when contacting a target. In some cases, the target is acne vulgaris. In some cases, the target is a biological marker on the surface of acne vulgaris. In some cases, the target is a lipid, a protein, a sugar, or a polysaccharide on the surface of acne vulgaris.

In some cases, the pH of the shelf-stable formulations and drug products provided herein is a pH that is effective to reduce or prevent degradation of the benzoyl peroxide to benzene. In some cases, the pH of the shelf-stable formulation is effective to prevent a concentration of benzene in the shelf-stable formulation from exceeding about 2 ppm. In some cases, the pH of the shelf-stable formulations and drug products provided herein is about 7 or less than about 7.

In some cases, the pH of the shelf-stable formulations and drug products provided herein is about 5 or less than about 5. In some cases, the pH of the shelf-stable formulations and drug products provided herein is about 3 or less than about 3. In some cases, the pH of the shelf-stable formulations and drug products provided herein is acidic.

In some cases, the shelf-stable formulations and drug products provided herein further comprise one or more active ingredients (e.g., in addition to the benzoyl peroxide). In some cases, the one or more active ingredients is or comprise an antibiotic. In some cases, the antibiotic is or comprises clindamycin, erythromycin, sulfur, or any combination thereof. In some cases, the one or more active ingredients is or comprises a retinoid or a retinoid-like compound. In some cases, the retinoid or retinoid-like compound is or comprises adapalene, tretinoin, or both. In some cases, the one or more active ingredient is or comprises an anti-inflammatory agent. In some cases, the anti-inflammatory agent is or comprises hydrocortisone, salicylic acid, niacinamide, or any combination thereof. In some cases, the anti-inflammatory agent is or comprises salicylic acid. In some cases, the one or more active ingredients is or comprises a diuretic. In some cases, the diuretic is or comprises spironolactone.

In various aspects, the shelf-stable formulations and drug products provided herein are topical formulations (e.g., for application to the skin or hair of a subject). In some cases, a shelf-stable formulation or drug product provided herein is a lotion, a cream, a gel, a jelly, a foam, a pad, a wipe, a solution, a bar, or a liquid. In some cases, a shelf-stable formulation or drug product provided herein is a face lotion, a face cream, a face gel, a face mask, a face wash, a soap, a foam cleanser, a face pad, a cleansing bar, a shaving cream, an emollient foam, a cleansing liquid, an exfoliant, a shampoo, or a hydrating wash.

In another aspect, the disclosure provides a formulation or a drug product comprising: a) an effective amount of benzoyl peroxide; and b) instructions or guidance to store the formulation or the drug product under conditions that prevent or reduce the degradation of the benzoyl peroxide to benzene. In some cases, the formulation or drug product comprises instructions or guidance to store the formulation or the drug product under conditions in which a concentration of benzene in the formulation or the drug product does not exceed about 2 ppm.

In some cases, the instructions or guidance may instruct or guide a consumer or end user to store the formulation or the drug product at a certain temperature to prevent or reduce degradation of the benzoyl peroxide to benzene. In some cases, the instructions or guidance may instruct or guide a consumer or end user to store the formulation or the drug product at a temperature at or below about 25° C. In some cases, the instructions or guidance may instruct or guide a consumer or end user to store the formulation or the drug product at a temperature at or below about 15° C. In some cases, the instructions or guidance may instruct or guide a consumer or end user to store the formulation or the drug product at a temperature at or below about 10° C. In some cases, the instructions or guidance may instruct or guide a consumer or end user to store the formulation or the drug product at a temperature at or below about 4° C. In some cases, the instructions or guidance may instruct or guide a consumer or end user to store the formulation or the drug product at a temperature at or below about 0° C. In some cases, the instructions or guidance may instruct or guide a consumer or end user to store the formulation or the drug product at a temperature at or below about −20° C. In some cases, the instructions or guidance may instruct or guide a consumer or end user to store the formulation or the drug product at a temperature at or below about −70° C. In some cases, the instructions or guidance instruct or guide a consumer or end user to store the formulation or the drug product away from exposure to light, radiation, heat, or any combination thereof.

In some cases, the conditions that prevent or reduce degradation of the benzoyl peroxide to benzene comprise storing the formulation or the drug product at a temperature from about −70° C. to about 25° C. In some cases, the conditions that prevent or reduce degradation of the benzoyl peroxide to benzene comprise storing the formulation or the drug product at a temperature from about 15° C. to about 25° C., from about 10° C. to about 15° C., from about 4° C. to about 10° C., from about 0° C. to about 4° C., from about −20° C. to about 0° C., or from about −70° C. to about −20° C. In some cases, the conditions that prevent or reduce degradation of the benzoyl peroxide to benzene comprise storing the formulation or the drug product at a temperature of at most about 25° C., at most about 15° C., at most about 10° C., at most about 4° C., at most about 0° C., at most about −20° C., or at most about −70° C. In some cases, the conditions that prevent or reduce degradation of the benzoyl peroxide to benzene comprise storing the formulation or the drug product at a temperature of at least about 25° C., at least about 15° C., at least about 10° C., at least about 4° C., at least about 0° C., at least about −20° C., or at least about −70° C.

In various aspects, the instructions or guidance may be provided (e.g., printed) on a label of the formulation or the drug product. The label may be provided on the packaging of the formulation or the drug product (e.g., such as on the container or vial containing the formulation or the drug product, or on a box or plastic wrap). In some cases, the instructions or guidance may be provided on an instruction sheet or manual provided separately from a label of the formulation or the drug product. In some cases, the instructions or guidance may be provided (e.g., verbally) by a physician or healthcare provider. In some cases, the instructions or guidance may be provided on a website or other electronic source.

In some aspects, the formulation or drug product provided herein may comprise any of the shelf-stable formulations provided herein (e.g., containing an effective amount of benzoyl peroxide). In some aspects, the formulation or the drug product may be in a packaging effective to reduce or prevent degradation of the benzoyl peroxide to benzene, as described herein.

In another aspect, the disclosure provides a formulation or a drug product comprising: a) an effective amount of benzoyl peroxide; and b) packaging containing the effective amount of benzoyl peroxide, wherein the packaging reduces or prevents the degradation of the benzoyl peroxide to benzene. In some cases, the packaging reduces or prevents the formation of benzene in the drug product. In some cases, the packaging prevents a concentration of benzene in the formulation or drug product from exceeding about 2 ppm.

In some cases, the packaging prevents or reduces exposure of the benzoyl peroxide to radiation (e.g., electromagnetic radiation). In some cases, the packaging prevents or reduces exposure of the benzoyl peroxide to light. In some cases, the packaging prevents or reduces exposure of the benzoyl peroxide to heat. In some cases, the packaging prevents or reduces significant variations in temperature. In some cases, the packaging maintains the temperature of the formulation within industry guidelines (e.g., according to United States Pharmacopeia (USP) <659>). In some cases, the packaging prevents the temperature of the formulation from exceeding industry guidelines (e.g., according to USP <659>).

In some aspects, the formulation or drug product provided herein may comprise any of the shelf-stable formulations provided herein (e.g., containing an effective amount of benzoyl peroxide). In some aspects, the formulation or drug product provided herein may comprise instructions or guidance to store the formulation or drug product under conditions that prevent or reduce the degradation of the benzoyl peroxide to benzene.

Further provided herein are drug lots comprising benzoyl peroxide that have high lot-to-lot consistency. In some aspects, a drug lot is provided comprising a plurality of drug products, each drug product comprising an effective amount of benzoyl peroxide, wherein no more than about 5% (e.g., no more than about 1%, no more than about 0.5%, no more than about 0.1%) of the plurality of drug products produce greater than 2 parts per million (ppm) benzene when the drug products are heated to about 37° C. for 7 days or more. In some aspects, a drug lot is provided comprising a plurality of drug products, each drug product comprising an effective amount of benzoyl peroxide, wherein no more than about 5% (e.g., no more than about 1%, no more than about 0.5%, no more than about 0.1%) of the plurality of drug products produce greater than 2 parts per million (ppm) benzene when the drug products are subjected to stability testing in accordance with guidelines from a regulatory agency.

Further provided herein are kits for e.g., generating or producing a compounded benzoyl peroxide drug product in accordance with embodiments provided herein. In some cases, the kit comprises a shelf-stable drug product or formulation comprising benzoyl peroxide contained in a first container or a plurality of first containers. The benzoyl peroxide may be in a substantially pure form (e.g., such as greater than 90% purity, greater than about 95% purity, greater than about 97% purity, greater than about 98% purity, greater than about 99% purity, greater than about 99.9% purity, or more), or may be in a formulation effective to reduce or prevent degradation of the benzoyl peroxide to benzene (e.g., as described herein). The benzoyl peroxide may be in the form of a crystalline powder. The kit may further comprise one or more additional ingredient contained in a second container or a plurality of second containers. The one or more additional ingredient may be selected from the group consisting of: a formulation, an inactive ingredient, an active ingredient (e.g., other than benzoyl peroxide), a solvent, a stabilizing agent, a capsule, a particle, and any combination thereof.

The kit may further comprise instructions (e.g., directed to a pharmacist, a medical professional, a consumer, etc.) for generating or producing the compounded benzoyl peroxide drug product. In some cases, the instructions may include instructions to combine the shelf-stable drug product or formulation comprising the benzoyl peroxide (e.g., from the first container) with the one or more additional ingredient (e.g., from the second container). In some cases, the shelf-stable drug product or formulation comprising the benzoyl peroxide (e.g., from the first container) may be added to the second container containing the one or more additional ingredient. In other cases, the one or more additional ingredient (e.g., from the second container) may be added to the first container containing the shelf-stable drug product or formulation comprising the benzoyl peroxide. In some cases, the kit may further comprise a third container for combining and/or mixing the shelf-stable drug product or formulation comprising the benzoyl peroxide with the one or more additional ingredient. The instructions may be provided on a packaging of the kit, on an instruction sheet or a manual provided with or within the kit, and/or may be provided separate from the kit (e.g., on a website or in electronic format).

In various aspects, when the shelf-stable drug product or formulation (e.g., contained with the first container(s)) is heated to 37° C. for 7 days or more, a concentration of benzene in the shelf-stable drug product or formulation is less than about 2 ppm, less than about 1 ppm, less than about 0.5 ppm, less than about 0.1 ppm, undetectable, or any combination thereof (e.g., as described herein). In various aspects, when the shelf-stable product or formulation (e.g., contained within the first container(s)) is subjected to stability testing following guidelines from a regulatory agency in a jurisdiction in which the shelf-stable drug product or formulation is to be registered or sold, the concentration of benzene in the shelf-stable drug product or formulation is less than about 2 ppm, less than about 1 ppm, less than about 0.5 ppm, less than about 0.1 ppm, undetectable, or any combination thereof (e.g., as described herein).

In various aspects, when the compounded benzoyl peroxide drug product (e.g., produced or generated with the contents of the kit, e.g., in accordance with instructions provided with the kit) is heated to 37° C. for 7 days or more, a concentration of benzene in the compounded benzoyl peroxide drug product is less than about 2 ppm, less than about 1 ppm, less than about 0.5 ppm, less than about 0.1 ppm, undetectable, or any combination thereof (e.g., as described herein). In various aspects, when the compounded benzoyl peroxide drug product (e.g., produced or generated with the contents of the kit, e.g., in accordance with instructions provided with the kit) is subjected to stability testing following guidelines from a regulatory agency in a jurisdiction in which the compounded benzoyl peroxide drug product or kit is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof (e.g., as described herein).

In various aspects, when a container (e.g., first container, second container), contents of a container, or a component of the kit is heated to 37° C. for 7 days or more, a concentration of benzene in the compounded benzoyl peroxide drug product is less than about 2 ppm, less than about 1 ppm, less than about 0.5 ppm, less than about 0.1 ppm, undetectable, or any combination thereof (e.g., as described herein). In various aspects, when a container (e.g., first container, second container), contents of a container, or a component of the kit is subjected to stability testing following guidelines from a regulatory agency in a jurisdiction in which the compounded benzoyl peroxide drug product or the kit is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof (e.g., as described herein).

In some cases, the compounded benzoyl peroxide drug product is stable for a period of time (e.g., does not form unacceptably high levels of benzene), after which point, the compounded benzoyl peroxide drug product may be unstable (e.g., forms unacceptably high levels of benzene). In various aspects, the compounded benzoyl peroxide drug product (e.g., produced or generated from the kit) is for use by an individual (e.g., subject, patient, end-user, consumer) within a period of time after generation of the compounded benzoyl peroxide drug product. In some cases, the compounded benzoyl peroxide drug product is stable for a period of time (e.g., does not form unacceptably high levels of benzene), after which point, the compounded benzoyl peroxide drug product may be unstable (e.g., forms unacceptably high levels of benzene). For example, a pharmacist may use the kit to generate or produce the compounded benzoyl peroxide drug product, and then provide the compounded benzoyl peroxide drug product to an individual for use within a period of time. In other cases, an individual (e.g., subject, patient, end-user, consumer) may use the kit to generate or produce the compounded benzoyl peroxide drug product for their own use within a period of time. In some cases, the period of time is selected from the group consisting of: 36 months or less, 24 months or less, 18 months or less, 12 months or less, 9 months or less, 6 months or less, 3 months or less, 90 days or less, 60 days or less, 45 days or less, 30 days or less, 15 days or less, 10 days or less, 7 days or less, 5 days or less, 72 hours or less, 48 hours or less, 24 hours or less, 12 hours or less, 1 hour or less, 30 minutes or less, 10 minutes or less, and 5 minutes or less. In some cases, the compounded benzoyl peroxide drug product is to be used immediately after the compounded benzoyl peroxide drug product is formed. In some cases, the compounded benzoyl peroxide drug product is for use by a human. In some cases, the compounded benzoyl peroxide drug product is for use by a non-human animal.

In various aspects, the kit may further comprise packaging which prevents or reduces the degradation of benzoyl peroxide to benzene (e.g., as described herein). For example, one or more of the first container(s), the second container(s), and/or third container(s), if used, may prevent or reduce the degradation of benzoyl peroxide to benzene. In various aspects, the kit may further comprise instructions to store the compounded benzoyl peroxide drug product, a container (e.g., first container, second container), or contents of a container, at a temperature selected from the group consisting of: at or below about 25° C., at or below about 20° C., at or below about 15° C., at or below about 10° C., at or below about 4° C., at or below about 0° C., at or below about −20° C., at or below about −70° C., and any combination thereof.

Further provided herein are compounded benzoyl peroxide products comprising: (a) an effective amount of benzoyl peroxide, wherein the benzoyl peroxide is in substantially pure form or is in a formulation effective to reduce or prevent degradation of the benzoyl peroxide to benzene; and (b) one or more ingredient. In some cases, the compounded benzoyl peroxide drug product is for use by an individual within a period of time after generation of the compounded benzoyl peroxide drug product. In some cases, the period of time is selected from the group consisting of: 36 months or less, 24 months or less, 18 months or less, 12 months or less, 9 months or less, 6 months or less, 3 months or less, 90 days or less, 60 days or less, 45 days or less, 30 days or less, 15 days or less, 10 days or less, 7 days or less, 5 days or less, 72 hours or less, 48 hours or less, 24 hours or less, 12 hours or less, 1 hour or less, 30 minutes or less, 10 minutes or less, and 5 minutes or less. In some cases, when the compounded benzoyl peroxide drug product is heated to 37° C., 50° C., or 70° C. for 7 days or more, a concentration of benzene in the compounded benzoyl peroxide drug product is less than about 2 ppm. In some cases, the one or more ingredient is selected from the group consisting of: a formulation, an inactive ingredient, an active ingredient other than benzoyl peroxide, a solvent, a stabilizing agent, a capsule, a particle, and any combination thereof. In some cases, the compounded benzoyl peroxide drug product is for use by an animal. In some cases, the benzoyl peroxide has a purity selected from the group consisting of: greater than about 90%, greater than about 95%, greater than about 97%, greater than about 98%, greater than about 99%, and greater than about 99.9%. In some cases, the benzoyl peroxide is in the form of a crystalline powder. In some cases, when the compounded benzoyl peroxide drug product is subjected to stability testing following guidelines from a regulatory agency in a jurisdiction in which the compounded benzoyl peroxide drug product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof. In some cases, the compounded benzoyl peroxide product further comprises packaging which prevents or reduces the degradation of benzoyl peroxide to benzene. In some cases, the compounded benzoyl peroxide product further comprises instructions to store the compounded benzoyl peroxide drug product at a temperature selected from the group consisting of: at or below about 25° C., at or below about 20° C., at or below about 15° C., at or below about 10° C., at or below about 4° C., at or below about 0° C., at or below about −20° C., at or below about −70° C., and any combination thereof. In some cases, when the compounded benzoyl peroxide product is subjected to stability testing following guidelines from a regulatory agency in a jurisdiction in which the compounded benzoyl peroxide product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof. In various aspects, methods of producing a compounded benzoyl peroxide product is provided (such as any compounded benzoyl peroxide product described herein) the method comprising: mixing or combining the benzoyl peroxide with one or more ingredient, thereby producing the compounded benzoyl peroxide product.

### Methods of the Disclosure

Further disclosed herein are methods of stabilizing formulations and drug products containing benzoyl peroxide. In some cases, the methods involve providing the benzoyl peroxide in a formulation (e.g., shelf-stable formulation) that prevents or reduces degradation or decomposition of the benzoyl peroxide to benzene. In some cases, the methods involve adding at least a stabilizing agent as described herein to the formulation in an amount effective to prevent or reduce degradation of the benzoyl peroxide to benzene.

In various aspects, the methods provided herein may involve providing or obtaining a formulation or a drug product comprising benzoyl peroxide that is identified as having or producing high levels of benzene. In some cases, the formulation or drug product may have or may form high levels of benzene under certain conditions (e.g., such as by heating to 37° C. for at least 7 days, as described herein). In some cases, benzene levels are considered to be high when they exceed 2 ppm. In some cases, benzene levels are considered to be high when they exceed a level permitted for use in a drug product by a regulatory agency (e.g., the FDA).

In various aspects, the methods further comprise formulating or re-formulating the formulation or drug product (e.g., identified as having or forming high levels of benzene) in a formulation that prevents or reduces the formation of benzene. In various aspects, the methods further comprise packaging or re-packaging the formulation or drug product (e.g., identified as forming high levels of benzene) in packaging that prevents or reduces the formation of benzene. In various aspects, the methods further comprise providing instructions or guidance to store the formulation or drug product under conditions that prevent or reduce the formation of benzene. In various aspects, the methods comprise any combination of formulating (or re-formulating), packaging (or re-packaging), and/or providing instructions or guidance, as described herein.

In some cases, the methods comprise adding at least one stabilizing agent or a plurality of stabilizing agents to the drug product, as provided herein. In some cases, the stabilizing agent(s) prevents or reduces degradation of the benzoyl peroxide to benzene. In some cases, the stabilizing agent(s) is provided in an amount effective to prevent or reduce the degradation of benzoyl peroxide to benzene. In some cases, the methods comprise adding a solvent to the formulation or the drug product as provided herein (e.g., in an amount effective to prevent or reduce the degradation of benzoyl peroxide to benzene). In some cases, the methods comprise providing one or more components (e.g., the benzoyl peroxide, the solvent, the stabilizing agent(s)) in an encapsulation to the formulation or drug product as provided herein (e.g., to prevent or reduce the degradation of benzoyl peroxide to benzene).

Further provided herein are methods of identifying a formulation or drug product comprising an effective amount of benzoyl peroxide as having or forming high levels of benzene as described herein. Generally, the methods involve providing or obtaining a formulation or drug product containing an effective amount of benzoyl peroxide, testing for the presence of benzene (e.g., employing one or more analytical techniques), and identifying the formulation or drug product as having or forming high levels of benzene. In some cases, the formulation or drug product is identified as having or forming high levels of benzene when the concentration of benzene present in the formulation or drug product is higher than a concentration approved by a regulatory agency for use in a drug product. In some cases, the formulation or drug product is identified as having or forming high levels of benzene when the concentration of benzene present in the formulation or drug product is higher than about 2 ppm. In some cases, one or more analytical techniques are employed to determine the concentration of benzene in the formulation or drug product. Non-limiting examples of analytical techniques that may be employed to determine the concentration of benzene in the formulation or drug product include gas chromatography/mass spectrometry (GC-MS), liquid chromatography/mass spectrometry (LC-MS), high-performance liquid chromatography (HPLC), nuclear magnetic resonance (NMR), gas chromatography with flame ionization detection (GC-FID), selected ion flow tube mass spectrometry (SIFT-MS), or any combination thereof.

In various aspects, the methods further comprise, after identifying the formulation or drug product as having or forming high levels of benzene, formulating or re-formulating the formulation or drug product in a formulation (e.g., shelf-stable formulation) that prevents or reduces degradation of benzoyl peroxide to benzene, as described herein. In various aspects, the methods further comprise, after identifying the formulation or drug product as having or forming high levels of benzene, stabilizing the benzoyl peroxide to prevent or reduce the degradation of benzoyl peroxide to benzene as described herein. In various aspects, the methods further comprise, after identifying the formulation or drug product as having or forming high levels of benzene, packaging or re-packaging the formulation or drug product in packaging that prevents or reduces the degradation of benzoyl peroxide to benzene as described herein. In various aspects, the methods further comprise, after identifying the formulation or drug product as having or forming high levels of benzene, providing instructions or guidance (e.g., to a purchaser, a retailer, a wholesaler, a packager, a shipper, a labeler, a consumer, an end user, or to any individual or entity involved in any part of the pharmaceutical supply chain) to store the formulation or drug product under conditions that prevent or reduce the degradation of benzoyl peroxide to benzene, as described herein.

In various aspects, the methods further comprise, prior to testing the benzene levels of the formulation or the drug product, subjecting the formulation or the drug product to one or more conditions, such as heating the formulation or drug product to 37° C. for at least 7 days, heating the formulation or drug product to 50° C. for at least 7 days, heating the formulation or drug product to 70° C. for at least 7 days, and/or subjecting the formulation or drug product to stability testing following guidelines from a regulatory agency, as described herein.

In some aspects, testing the formulation or the drug product comprises testing the formulation or the drug product for the presence of benzene at a first time point and at a second time point. In some cases, a concentration of benzene in the formulation or the drug product at the second time point is greater than at the first time point. In some cases, at the first time point, the formulation or the drug product has a concentration of benzene that is less than about 2 ppm, and at the second time point, the formulation or the drug product has a concentration of benzene that is greater than about 2 ppm.

In some cases, an effective amount of benzoyl peroxide is an amount from about 2.5% w/v to about 10% w/v. In some cases, an effective amount of benzoyl peroxide is about 2.5% w/v. In some cases, an effective amount of benzoyl peroxide is about 2.7% w/v. In some cases, an effective amount of benzoyl peroxide is about 3% w/v. In some cases, an effective amount of benzoyl peroxide is about 4% w/v. In some cases, an effective amount of benzoyl peroxide is about 4.4% w/v. In some cases, an effective amount of benzoyl peroxide is about 4.5% w/v. In some cases, an effective amount of benzoyl peroxide is about 5% w/v. In some cases, an effective amount of benzoyl peroxide is about 5.3% w/v. In some cases, an effective amount of benzoyl peroxide is about 6% w/v. In some cases, an effective amount of benzoyl peroxide is about 7% w/v. In some cases, an effective amount of benzoyl peroxide is about 7.5% w/v. In some cases, an effective amount of benzoyl peroxide is about 8% w/v. In some cases, an effective amount of benzoyl peroxide is about 9.8% w/v. In some cases, an effective amount of benzoyl peroxide is about 10% w/v. In some cases, an effective amount of benzoyl peroxide is an amount approved (e.g., for use in or on a subject) by a regulatory agency.

In some cases, the drug product is a drug product approved for use on a human by a regulatory agency. In some cases, the drug product is a drug product approved to treat a condition (e.g., a skin condition) in a human by a regulatory agency. In some cases, the drug product is a drug product marketed and sold commercially, e.g., for the treatment of a condition (e.g., a skin condition). In some cases, the skin condition is acne vulgaris. In some cases, the regulatory agency is selected from the group consisting of: the United States Food and Drug Administration (FDA), the European Medicines Agency (EMA), Japan's Pharmaceutical and Medical Devices Agency (PMDA), Health Canada, the Swiss Agency for Therapeutic Products (Swissmedic), and Australia's Therapeutic Goods Administration (TGA). In some cases, the drug product is a topical formulation of the benzoyl peroxide. In some cases, the drug product is a drug product for veterinary use (e.g., on or in a non-human animal, e.g., a dog, a cat, livestock (e.g., a cow, a sheep, a horse, a pig).

In some cases, the drug product further comprises one or more active ingredients (e.g., in addition to the benzoyl peroxide). In some cases, the one or more active ingredients is or comprises an antibiotic. In some cases, the antibiotic is or comprises clindamycin, erythromycin, sulfur, or any combination thereof. In some cases, the one or more active ingredients is or comprises a retinoid or a retinoid-like compound. In some cases, the retinoid or retinoid-like compound is or comprises adapalene, tretinoin, or both. In some cases, the one or more active ingredients is or comprises an anti-inflammatory agent. In some cases, the anti-inflammatory agent is or comprises hydrocortisone, salicylic acid, niacinamide, or any combination thereof. In some cases, the one or more active ingredients is or comprises a diuretic. In some cases, the diuretic is or comprises spironolactone.

In some cases, the drug product is selected from the group consisting of: 111MedCo®, Acne-Clear®, AcneFree®, Acne Wipeout®, Acnigel®, Anti Bac®, Asepxia®, Ben-Aqua®, Benzac®, Benzagel®, Benzashave®, BenzEFoam®, Benziq®, Benzoil®, Bioelements®, Binora®, Botanic Tree®, Brevoxyl®, CeraVe®, Claridad®, Clean & Clear®, Clear By Design®, Clearasil®, Clearplex®, Clearskin®, Clinac BPO®, CVS®, CVS Health®, Daylogic®, Del-Aqua®, Dermaquest®, Desquam®, Differin®, Dr. Song®, Dr. Zenovia®, Epionce®, Equate®, Equate Beauty®, Ethexderm BPW®, Fostex®, Glo Skin Beauty®, Glossier®, Glytone®, Harris Pharmaceutical®, Humane®, Inova®, Jan Marini Skin Research®, Johnson&Johnson®, Kate Sommerville®, Kroger®, La Roche-Posay®, Lavoclen®, Loroxide®, MDacne®, NeoBenz®, Neutrogena®, Oscion®, Oxy 10®, Pacnex®, PanOxyl®, Paula's Choice®, PCA Skin®, Peroderm®, Persa-Gel®, Peroxin A®, Perrigo®, Persa-Gel®, Proactiv®, ProactivMD®, Proactiv+®, Proactiv Solution®, RB Health®, Replenix Acne®, Rugby Laboratories®, SalSphere®, Salvona®, Seba-Gel®, Signature Caret, Skin Bar MD®, SLMD Sandra Lee MD®, SMACNE®, Solimo®, Soluclenz®, Sonya Dakar®, Taro Pharmaceuticals, Theroxide®, Touch®, Triaz®, Tula Skincare®, Up&Up®, Vanoxide®, Vivant Skincare®, Walgreens®, Zaclir®, Zapzyt®, Zeroxin®, Zit Stick®, ZoDerm®, Acanya®, Bencort®, Benzaclin®, Benzamycin®, Duac®, Epiduo®, Face Up®, NuOx®, Sulfoxyl®, and Vanoxide-HC®.

In some cases, when the formulation or drug product is identified as having or forming high levels of benzene, the method further comprises indicating that the formulation or drug product should be formulated or re-formulated in a formulation that prevents or reduces degradation of the benzoyl peroxide to benzene. In some cases, when the formulation or drug product is identified as having or forming high levels of benzene, it is indicated that the formulation or drug product should be formulated or re-formulated in a formulation that prevents or reduces degradation of the benzoyl peroxide to benzene.

In some cases, when the formulation or the drug product is identified as having or forming high levels of benzene, the method further comprises indicating that the formulation or the drug product should be provided with instructions or guidance to store the formulation, the drug product, or an ingredient used to manufacture the drug product, under conditions (e.g., lower temperature) that prevent or reduce degradation of the benzoyl peroxide to benzene. In some cases, when the formulation or the drug product is identified as having or forming high levels of benzene, it is indicated that the formulation or the drug product should be stored under conditions (e.g., lower temperature) that prevent or reduce degradation of the benzoyl peroxide to benzene.

In some cases, when the formulation or the drug product is identified as having or forming high levels of benzene, the method further comprises indicating that the formulation or the drug product should be packaged or re-packaged in packaging that prevents or reduces degradation of the benzoyl peroxide to benzene. In some cases, when the formulation or the drug product is identified as having or forming high levels of benzene, it is indicated that the formulation or the drug product should be packaged or re-packaged in packaging that prevents or reduces degradation of the benzoyl peroxide to benzene.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### EXAMPLES

### Example 1. Protocols for Analyzing Benzene Levels in Formulations Containing Benzoyl Peroxide

### Materials and Methods

Agilent 7890B Gas Chromatography (GC) system equipped with 7697A headspace autosampler coupled with 5977B Mass Selective Detector (MS) was used for sample analysis, and a DB-Select 624 UI, 60 m×0.32 mm×1.8 μm GC column (Agilent Technology, Santa Clara, CA) was used to separate benzene from other compounds. Dimethyl sulfoxide (DMSO, GC Headspace Grade) was used for sample preparation (Thermo Fisher Scientific, Waltham, MA). All certified reference standards, solution, and buffer reagents including sodium citrate dihydrate, citric acid, tris, and glycerol were sourced from Sigma-Aldrich, St. Louis, MO.

### Standard and Sample Preparation

Samples were prepared in 20 mL glass GC headspace vials to a total of 5 mL volume. Benzoyl peroxide products were brought to room temperature, gently shaken to homogenize and dispensed into the GC headspace vials at approximate 500 mg and weighed. Notably, products incubated at 50° C. changed consistency over time and gentle shaking helped to ensure homogenous sample collection. Products analyzed with addition of antioxidant were prepared in the same way with 50 mg of dry antioxidant powder added (10% w/v), mixed manually, and crimp sealed for incubation. For analysis, 4.5 mL of DMSO was added to make up the final volume to approximately 5 mL, crimp sealed, and gently vortexed to mix.

For benzoyl peroxide stability studies in glycerol, benzoyl peroxide was prepared in a stock suspension of 20% w/v in glycerol and mixed 1:1 with 20% w/v suspensions or v/v solutions of antioxidants in glycerol. For pH experiments, the same stock suspension of benzoyl peroxide was mixed 1:1 with a 50% glycerol solution with 200 mM buffering agent (Citrate buffer pH 3.0, Tris Buffer pH 7.0, or Tris Buffer pH 9.0) for final 75% glycerol solution. The final volume of all reactions was 100 μL in GC headspace vials with 10% solution benzoyl peroxide (10 mg) and 10% antioxidant or 100 mM buffering agent. Vials were immediately crimp sealed for incubation. For analysis, 4.9 mL of DMSO was added to make up the final volume to approximate 5 mL, crimp sealed, and gently vortexed to mix.

Benzene standard was diluted in DMSO for an 11-point calibration ranging from 10 ng/vial to 100 μg/vial. The coefficient of determination R² were greater than 0.999. Five (5) mL of DMSO was used for negative control samples.

### Incubation

Benzoyl peroxide products in original packaging were incubated for up to one month at 25° C., 37° C., or 50° C. and sampled periodically. Benzoyl peroxide and products with antioxidant additions or buffering agent additions were prepared and incubated in crimp sealed GC headspace vials.

### Analytical Methods

The method USP <467> Residual Solvents Procedure A was modified from flame ionization detection (FID) to mass spectrometry (MS) detection for benzene in benzoyl peroxide products. The gas chromatography (GC) methods were also modified to allow shorter run time and the method was validated for addition of up to 500 mg benzoyl peroxide product or 200 mL glycerol. Identification of benzene was based on the retention time matching to certified reference standards and mass spectral m/z matching to benzene. Lower limit of detection (LLOD) was 10 ng (equivalent to 0.02 ppm in hand benzoyl peroxide products) and LLOQ was 25 μg. The measurement uncertainty was determined to be 20%.

Table 1 summerizes the major instrumental parameters used for analysis of benzene.

Headspace Sampling Method

| Headspace Autosampler | | | GC | | | MS | | |
|---|---|---|---|---|---|---|---|---|
| Oven temperature | 37° | C. | Carrier gas | Helium | | Source Temp | 230° | C. |
| (Temp) | | | | | | | | |
| Loop Temp | 55° | C. | Inlet Temp | 220° | C. | Quad Temp | 150° | C. |

| | | | | | | | |
|---|---|---|---|---|---|---|---|
| Transfer line Temp | 175° | C. | Column flow | 2 | mL/min | Acquisition type | SIM |

| Vial equilibration | 20 | min | Split ratio | 5:1 | Gain factor | 1.2 | |
|---|---|---|---|---|---|---|---|
| Injection Duration | 1 | min | Oven Temp | 60° C. | Solvent delay | 3 | min |
| | | | Gradient | (12 min) > | | | |
| | | | | 240° C. at | | | |
| | | | | 40° C./min | | | |
| | | | | (2 min) | | | |

| | | | | | |
|---|---|---|---|---|---|
| Vial shaking | 71 | shakes/min | GC run time | 18.5 | min |

| | | |
|---|---|---|
| Fill pressure | 15 | psi |

### Example 2. Various FDA-Approved Drug Products Containing Benzoyl Peroxide Form Unacceptably High Levels of Benzene

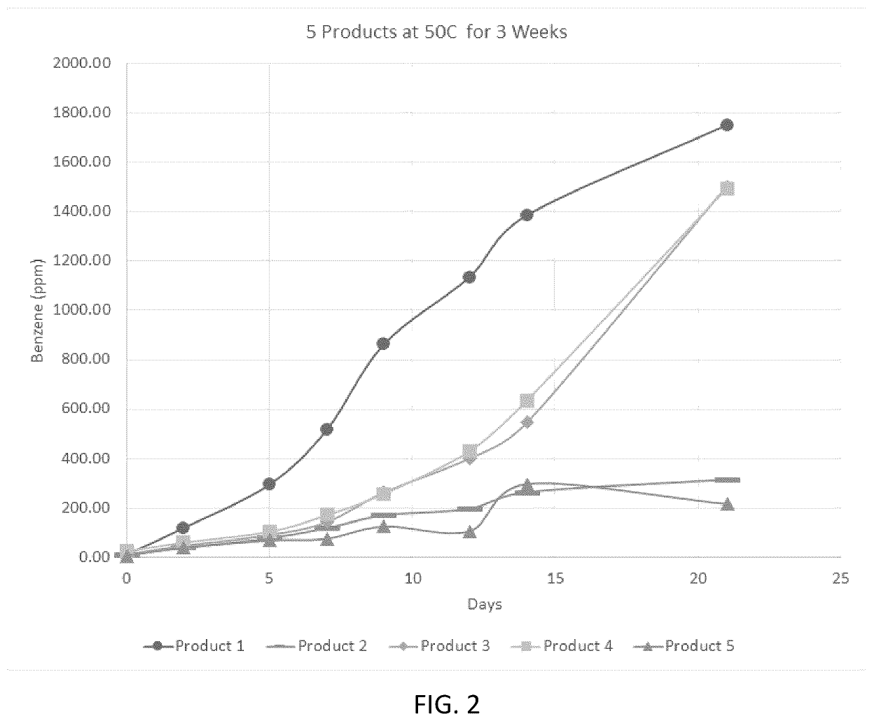

Five FDA-approved drug products containing benzoyl peroxide were selected for testing. These drug products were commercially-available topical formulations of benzoyl peroxide marketed for the treatment of skin conditions (e.g., acne vulgaris) in humans. The drug products were incubated at 50° C. for up to three weeks. Benzene levels in each drug product were tested at various time points according to the protocols described in Example 1. FIG. 2 depicts the levels of benzene (ppm) formed in the drug products at various time points. Unexpectedly and surprisingly, benzene increased to levels that were well above the current, least strict FDA limit for benzene in drug products (2 ppm), even after incubation at 50° C. for only a few days, with some drug products reaching benzene levels that were almost 1000-times the FDA limit.

### Example 3. Antioxidants Prevent or Reduce Formation of Benzene in Benzoyl Peroxide-Containing Formulations

10% w/v benzoyl peroxide formulations were prepared. Various antioxidants were added to the formulations (10% w/v butylated hydroxytoluene (BHT), 10% w/v vitamin E acetate, or 5% w/v tert-butyl hydroquinone (TBHQ)+5% w/v BHT) to test whether these antioxidants could prevent or reduce formation of benzene under certain conditions. The various formulations were incubated at either 37° C. for up to two weeks or at 50° C. for up to two weeks. Benzene levels in each formulation were tested at various time points according to the protocols described in Example 1.

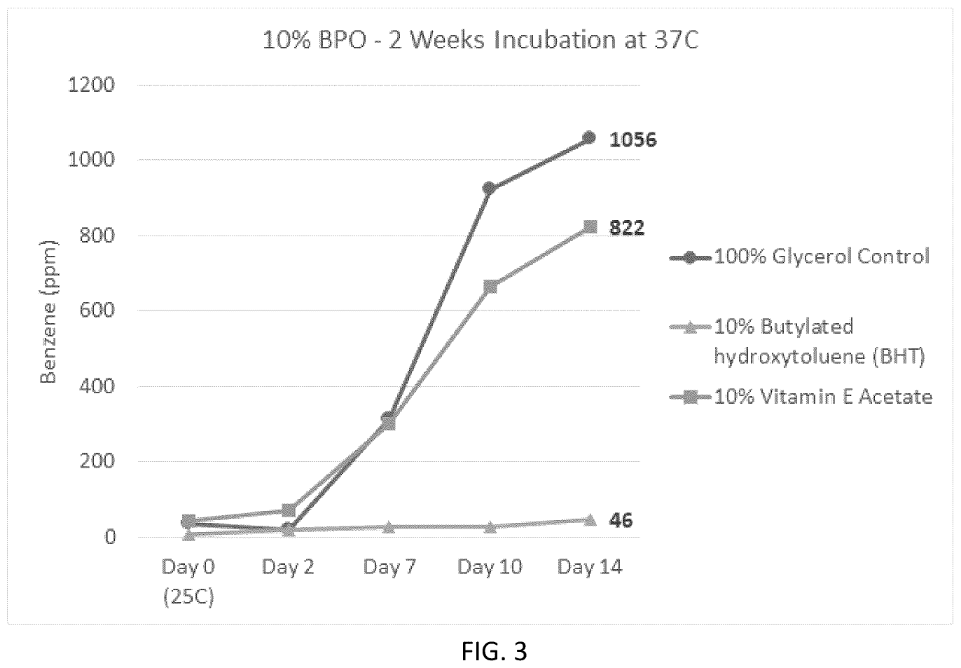

FIG. 3 depicts the levels of benzene (ppm) formed in the formulations at various time points when incubated at 37° C. FIG. 3 demonstrates that both BHT and vitamin E acetate reduced the levels of benzene when the formulations were incubated at 37° C. for up to two weeks, with BHT showing the most dramatic reduction.

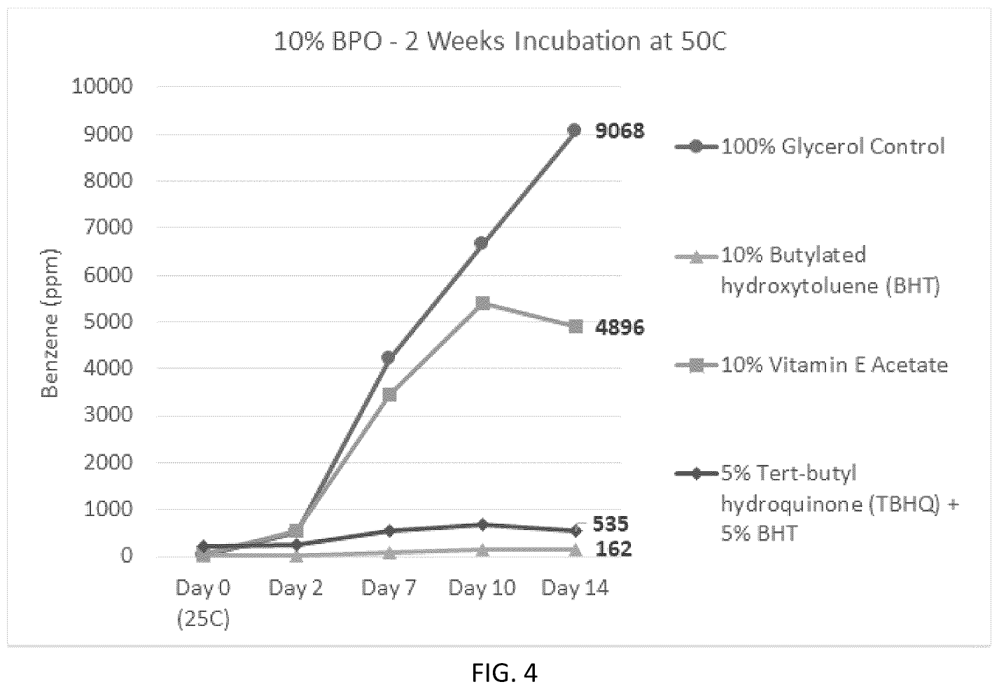

FIG. 4 depicts the levels of benzene (ppm) formed in the formulations at various time points when incubated at 50° C. FIG. 4 demonstrates that BHT, vitamin E acetate, and a combination of BHT and TBHQ, reduced the levels of benzene when the formulations where stored at 50° C. for up to two weeks, with BHT and a combination of BHT and TBHQ showing the most dramatic reduction.

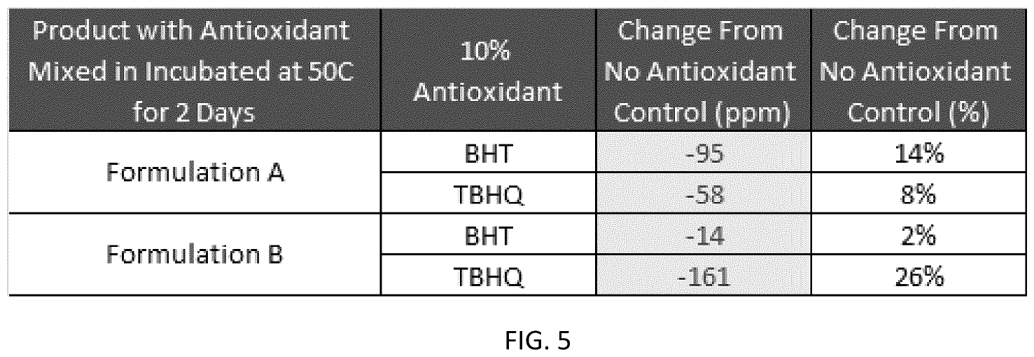

FIG. 5 depicts the levels of benzene (ppm) formed in two different FDA-approved drug products containing benzoyl peroxide and an antioxidant to stabilize the benzoyl peroxide, after incubation at 50° C. for 2 days. FIG. 5 demonstrates that both BHT and TBHQ stabilize benzoyl peroxide and reduce the degradation of benzoyl peroxide to benzene, when added to commercially-available drug products approved by the FDA.

The data taken together demonstrates that various antioxidants may be effective at reducing or preventing benzene levels in formulations containing benzoyl peroxide.

### Example 4. Various Solvents Prevent or Reduce Formation of Benzene in Benzoyl Peroxide-Containing Formulations

10% w/v benzoyl peroxide formulations were prepared. Various solvents were added to the formulations (water, dimethylsulfoxide (DMSO), glycerol, N-methyl-2-pyrrolidone (NMP)) to test whether these solvents could prevent or reduce formation of benzene under certain conditions. The various formulations were incubated at either 37° C. for up to two weeks or at 50° C. for up to two weeks. Benzene levels in each formulation were tested at various time points according to the protocols described in Example 1.

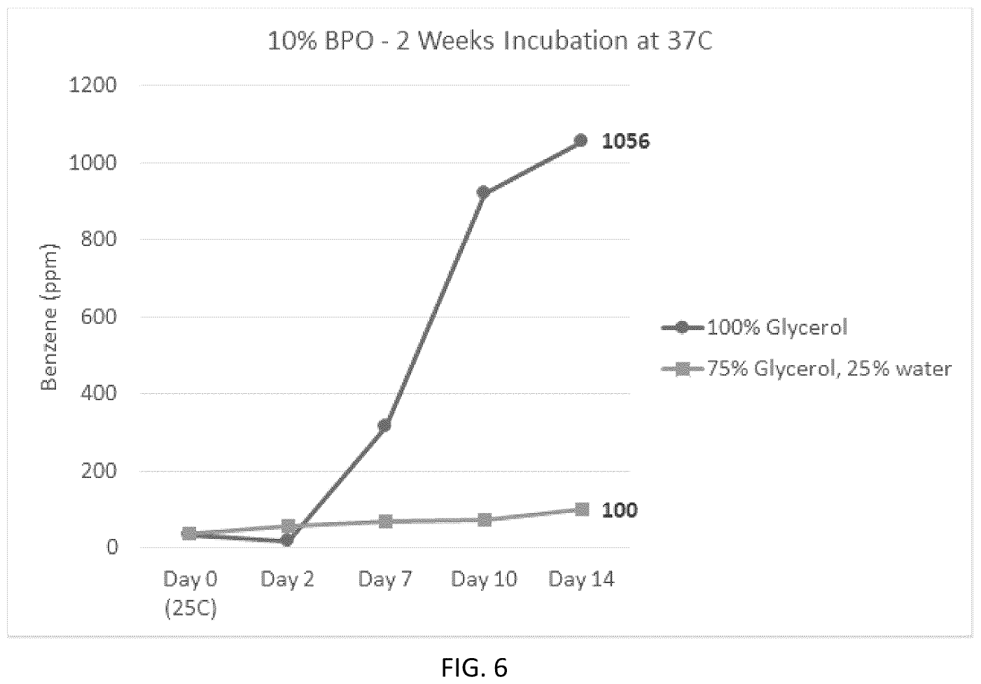

FIG. 6 depicts the levels of benzene (ppm) formed in the formulations at various time points when incubated at 37° C. FIG. 6 demonstrates that a formulation containing 25% v/v water and 75% v/v glycerol dramatically reduced the levels of benzene formed as compared to a 100% v/v glycerol control.

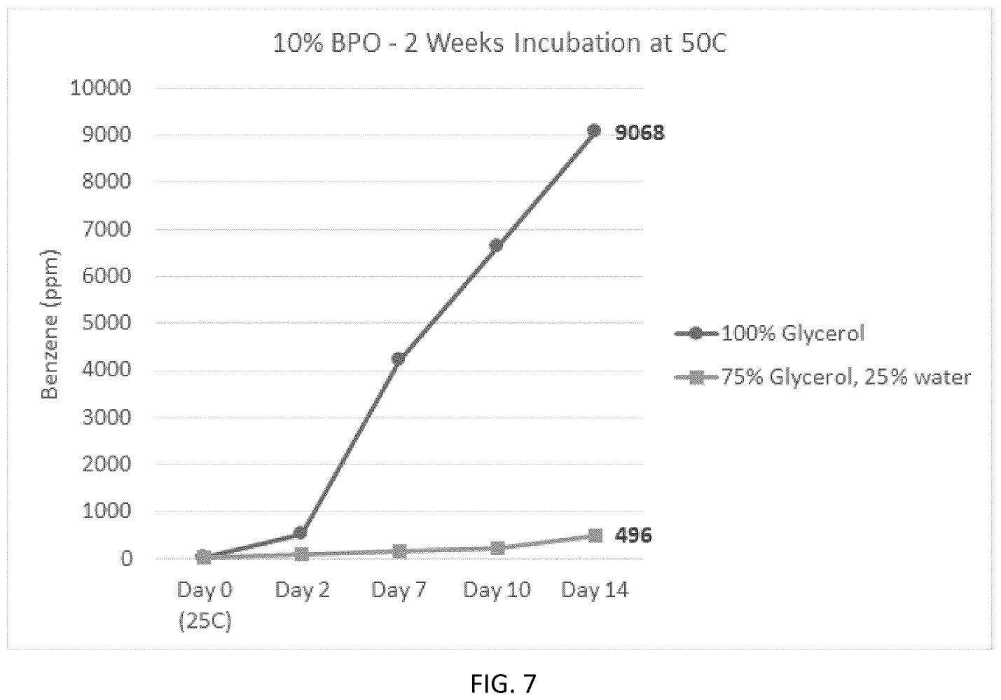

FIG. 7 depicts the levels of benzene (ppm) formed in the formulations at various time points when incubated at 50° C. FIG. 7 demonstrates that a formulation containing 25% v/v water and 75% v/v glycerol dramatically reduced the levels of benzene formed as compared to a 100% v/v glycerol control.

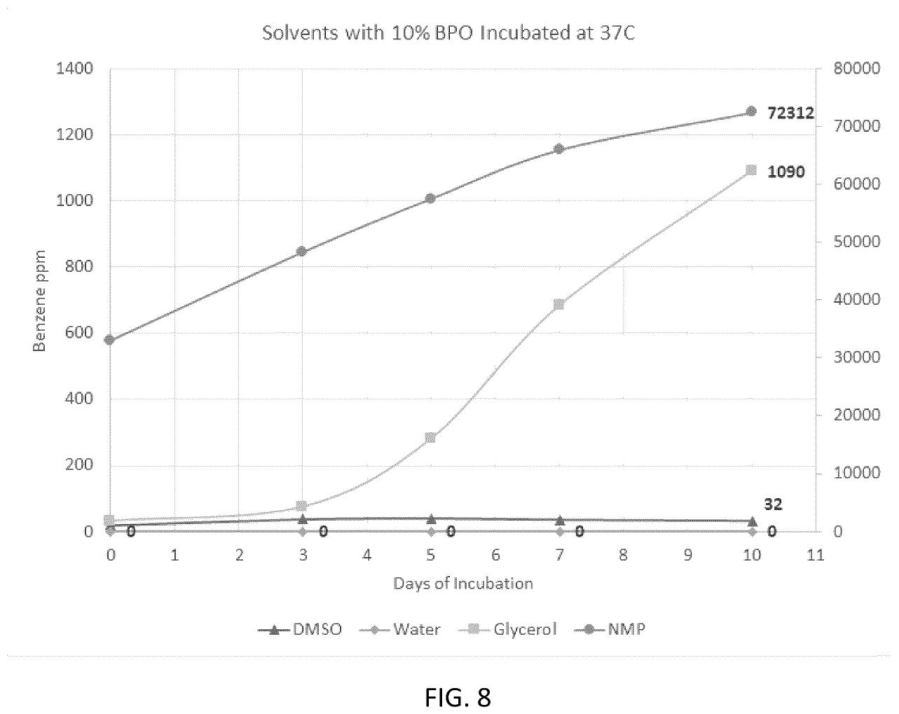

FIG. 8 depicts the levels of benzene (ppm) formed in the formulations at various time points when incubated at 37° C. FIG. 8 demonstrates that formulations containing DMSO or water showed the most dramatic reduction in, or complete prevention of, benzene levels, as compared to the formulations containing glycerol or NMP.

The data taken together demonstrates that various solvents may be effective at reducing or preventing benzene levels in formulations containing benzoyl peroxide.

### Example 5. Lower pH Levels May Prevent or Reduce Formation of Benzene in Benzoyl Peroxide-Containing Formulations

10% w/v benzoyl peroxide formulations were prepared in 75% v/v glycerol, with or without various buffers to alter the pH. Formulations at pH 3, pH 7, and pH 9 were tested, relative to a no buffer control. The various formulations were incubated at either 37° C. for up to 10 days or at 50° C. for up to 10 days. Benzene levels in each formulation were tested at various time points according to the protocols described in Example 1.

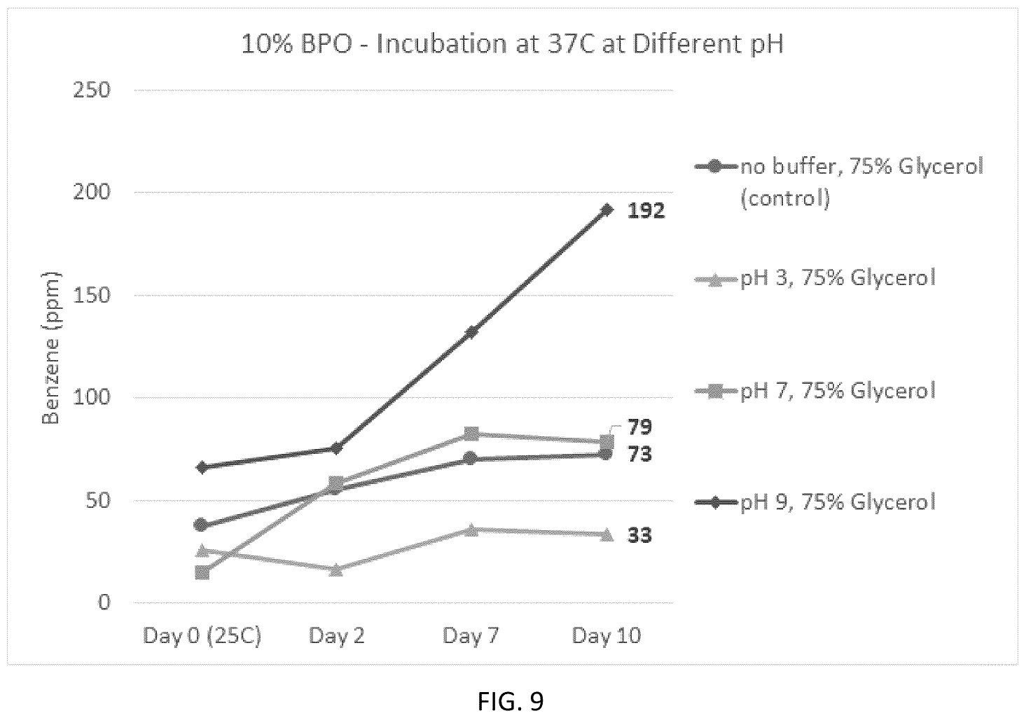

FIG. 9 depicts the levels of benzene (ppm) formed in the formulations at various time points when incubated at 37° C. FIG. 9 demonstrates that lowering the pH dramatically reduced the levels of benzene formed as compared to a no buffer control.

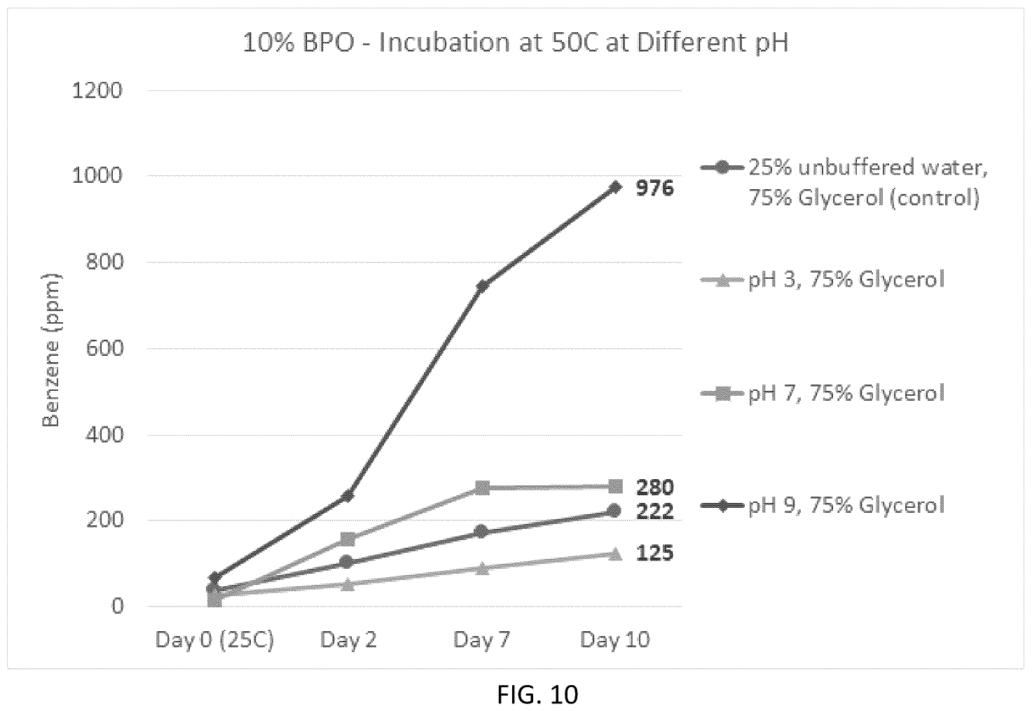

FIG. 10 depicts the levels of benzene (ppm) formed in the formulations at various time points when incubated at 50° C. FIG. 10 demonstrates that lowering the pH dramatically reduced the levels of benzene formed as compared to a no buffer control.

The data taken together demonstrates that lower pH may be effective at reducing or preventing benzene levels in formulations containing benzoyl peroxide.

### Example 6. Crystalline Benzoyl Peroxide May Form Low Levels of Benzene

Crystalline benzoyl peroxide was obtained and incubated at 50° C. for up to 12 days. Benzene levels were tested at various time points according to the protocols described in Example 1.

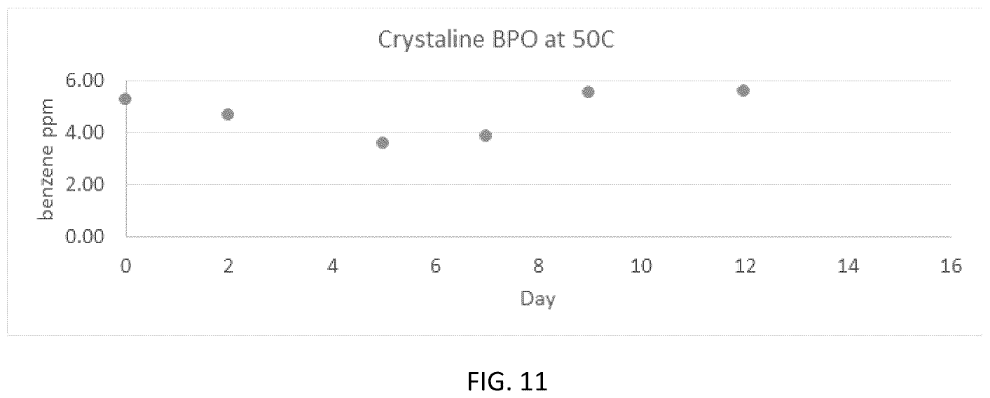

FIG. 11 depicts the levels of benzene (ppm) formed at various time points when the crystalline benzoyl peroxide was incubated at 50° C. FIG. 11 demonstrates that crystalline benzoyl peroxide may be protected from forming benzene levels under increased temperature conditions.

### Example 7. Commercially Available Encapsulated Benzoyl Peroxide Products Form Unacceptably High Levels of Benzene

A commercially-available encapsulated benzoyl peroxide was obtained and incubated at 50° C. for up to 8 days. Benzene levels were tested at various time points according to the protocols described in Example 1.

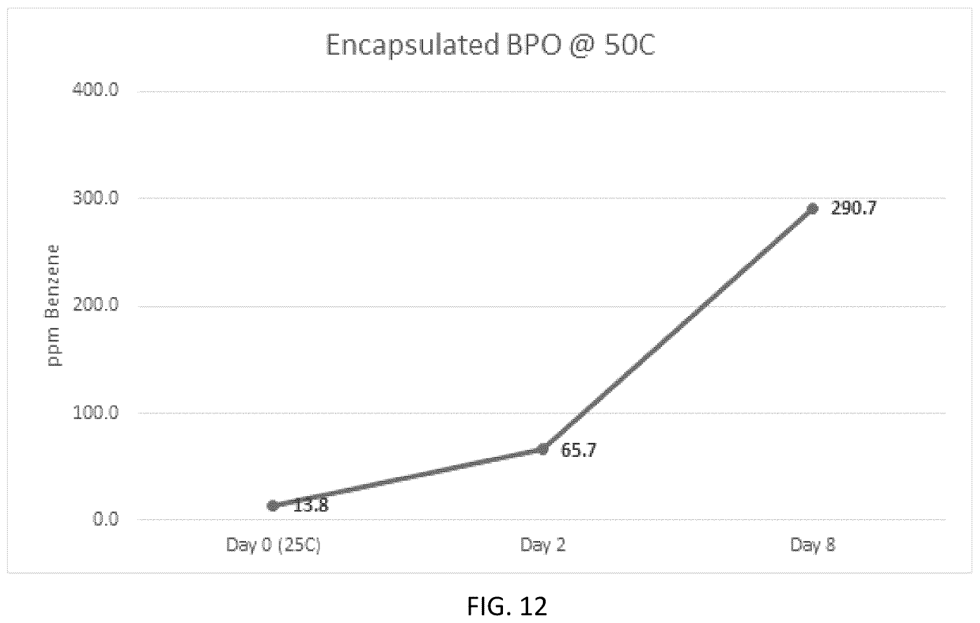

FIG. 12 depicts the levels of benzene (ppm) formed at various time points when the encapsulated benzoyl peroxide was incubated at 50° C. FIG. 12 demonstrates that at least one commercially-available encapsulated benzoyl peroxide may form unacceptable high levels of benzene under certain conditions. This further demonstrates that at least one commercial process for encapsulating benzoyl peroxide is insufficient to meaningfully prevent the degradation of benzoyl peroxide into benzene.

### Example 8. Increasing Water Content and Reducing pH Levels Stabilizes Commercially Available Benzoyl Peroxide Formulations and Prevents Production of High Benzene Levels

Four commercially-available benzoyl peroxide formulations were obtained and incubated at 50° C. for up to 14 days. The commercially-available benzoyl peroxide formulations were also re-formulated by increasing the water content and reducing pH levels (the concentration of benzoyl peroxide remained the same) to assess the effect of water content and pH levels on benzene formation. Benzene levels were tested at various time points according to the protocols described in Example 1.

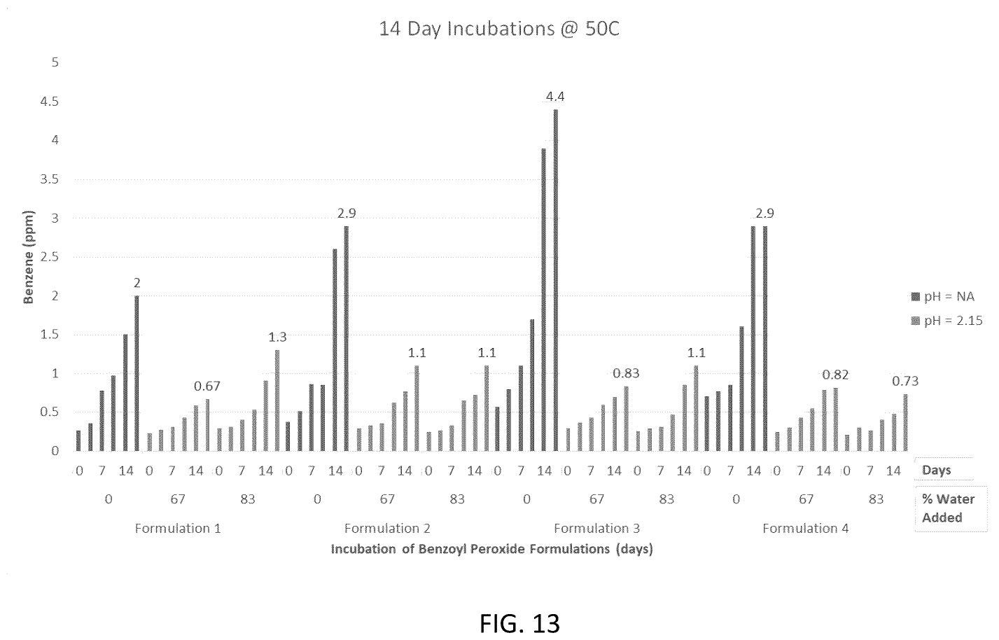

FIG. 13 depicts the levels of benzene (ppm) formed at various time points when the commercially-available formulations and the re-formulated, stabilized formulations were incubated at 50° C. FIG. 13 demonstrates that all four commercially-available formulations formed unacceptably high levels of benzene under these conditions. All four re-formulated, stabilized formulations (e.g., increased water content, decreased pH levels) formed benzene under the same conditions that were below the levels acceptable to regulatory agencies. This example demonstrates that unstable formulations of benzoyl peroxide may be re-formulated into shelf-stable formulations (e.g., by increasing water content and/or reducing pH levels).

What is claimed is:

1. A kit comprising:
(a) a first container or a plurality of first containers containing therein benzoyl peroxide, wherein the benzoyl peroxide is in substantially pure form or is in a formulation effective to reduce or prevent degradation of the benzoyl peroxide to benzene;
(b) a second container or a plurality of second containers containing therein at least one ingredient; and
(c) instructions for combining the benzoyl peroxide with the at least one ingredient to generate a compounded benzoyl peroxide drug product.

2. The kit of claim 1, wherein, when the first container or plurality of first containers is heated to 70° C. for 7 days or more, a concentration of benzene in the formulation is less than about 2 parts per million (2 ppm).

3. The kit of claim 1, wherein, when the compounded benzoyl peroxide drug product is heated to 70° C. for 7 days or more, a concentration of benzene in the compounded benzoyl peroxide drug product is less than about 2 ppm.

4. The kit of claim 1, wherein the at least one ingredient is selected from the group consisting of: a formulation, an inactive ingredient, an active ingredient other than benzoyl peroxide, a solvent, a stabilizing agent, a capsule, a particle, and any combination thereof.

5. The kit of claim 4, wherein the active ingredient other than benzoyl peroxide is selected from the group consisting of: an antibiotic, a retinoid or retinoid-like compound, an anti-inflammatory agent, a diuretic, and any combination thereof.

6. The kit of claim 1, wherein the compounded benzoyl peroxide drug product is for use by an individual within a period of time after generation of the compounded benzoyl peroxide drug product.

7. The kit of claim 6, wherein the period of time is selected from the group consisting of: 36 months or less, 24 months or less, 18 months or less, 12 months or less, 9 months or less, 6 months or less, 3 months or less, 90 days or less, 60 days or less, 45 days or less, 30 days or less, 15 days or less, 10 days or less, 7 days or less, 5 days or less, 72 hours or less, 48 hours or less, 24 hours or less, 12 hours or less, 1 hour or less, 30 minutes or less, 10 minutes or less, and 5 minutes or less.

8. The kit of claim 1, wherein the compounded benzoyl peroxide drug product is for use by an animal.

9. The kit of claim 1, wherein the compounded benzoyl peroxide drug product is for use by a human.

10. The kit of claim 1, wherein the benzoyl peroxide in substantially pure form has a purity selected from the group consisting of: greater than about 90%, greater than about 95%, greater than about 97%, greater than about 98%, greater than about 99%, and greater than about 99.9%.

11. The kit of claim 1, wherein the benzoyl peroxide is in the form of a crystalline powder.

12. The kit of claim 1, wherein, when the compounded benzoyl peroxide drug product is subjected to stability testing following guidelines from a regulatory agency in a jurisdiction in which the compounded benzoyl peroxide drug product is to be registered or sold, the concentration of benzene in the compounded benzoyl peroxide drug product is less than about 2 ppm, less than about 1 ppm, less than about 0.5 ppm, less than about 0.1 ppm, undetectable, or any combination thereof.

13. The kit of claim 1, wherein the kit further comprises packaging which prevents or reduces the degradation of benzoyl peroxide to benzene.

14. The kit of claim 13, wherein the packaging prevents or reduces exposure of the benzoyl peroxide to light.

15. The kit of claim 1, further comprising instructions to store the first container or plurality of first containers, the second container or plurality of second containers, the compounded benzoyl peroxide drug product, or any combination thereof, at a temperature selected from the group consisting of: at or below about 25° C., at or below about 20° C., at or below about 15° C., at or below about 10° C., at or below about 4° C., at or below about 0° C., at or below about −20° C., at or below about −70° C., and any combination thereof.

16. The kit of claim 1, wherein an amount or concentration of benzoyl peroxide in the compounded benzoyl peroxide drug product is an amount or concentration of benzoyl peroxide effective for treating a skin condition in a subject.

17. The kit of claim 16, wherein the concentration of benzoyl peroxide in the compounded benzoyl peroxide drug product is from about 2.5% w/w to about 10% w/w.

18. The kit of claim 17, wherein the skin condition is acne vulgaris.

19. The kit of claim 1, wherein the compounded benzoyl peroxide drug product is a lotion, a cream, a gel, a jelly, a foam, a pad, a wipe, a solution, a bar, or a liquid.

20. A method of producing a compounded benzoyl peroxide drug product using the kit of claim 1, the method comprising: mixing or combining an amount of the benzoyl peroxide from the first container or plurality of first containers with an amount of the at least one ingredient from the second container or plurality of second containers to form the compounded benzoyl peroxide drug product, such that the benzoyl peroxide is present in the compounded benzoyl peroxide drug product in an amount or concentration effective to treat a disease, a disorder, or a condition of a subject.
